(19) United States
(12) Patent Application Publication
(10) Pub. No.: US 2021/0308286 A1
(43) Pub. Date: Oct. 7, 2021

(54) LONG-CIRCULATING PSMA-TARGETED PHOTOTHERANOSTIC AGENT

(71) Applicants: The Johns Hopkins University, Baltimore, MD (US); University Health Network, Toronto (CA)

(72) Inventors: Martin G. Pomper, Baltimore, MD (US); Ronnie C. Mease, FAIRFAX, VA (US); Ying Chen, Luhterville-Timonium, MD (US); Sangeeta Ray, Ellicott City, MD (US); Juan Chen, Toronto (CA); Marta Overchuk, TORONTO (CA); Kara Harmatys, Toronto (CA); Gang Zheng, Toronto (CA)

(21) Appl. No.: 17/264,220

(22) PCT Filed: Jul. 30, 2019
(86) PCT No.: PCT/US2019/044075

§ 371 Date: Jan. 28, 2021

Related U.S. Application Data
(60) Provisional application No. 62/711,930, filed on Jul. 30, 2018.

(51) Int. Cl. A61K 51/08; A61K 41/00; A61P 35/00; A61K 49/00; A61K 47/65

(52) U.S. Cl. CPC A61K 51/088; A61K 41/0071; A61P 35/00; A61K 41/0076; A61K 49/0036; A61K 47/65; A61K 49/0056

(57) ABSTRACT

Theranostic probes comprising a porphyrin-based photosensitizer, a D-peptide linker, and a urea-based PSMA-targeting ligand and methods of their use for treating and/or imaging PMSA-expressing tumors are disclosed.

### FEDERALLY SPONSORED RESEARCH OR DEVELOPMENT

This invention was made with government support under EB024495, CA202199, CA184228, and CA134675 awarded by the National Institutes of Health. The government has certain rights in the invention.

### BACKGROUND

In the era of precision medicine, significant efforts have been devoted toward the development of new theranostic strategies that allow simultaneous detection, delineation, and treatment of tumors. Recently photodynamic therapy (PDT) has emerged as a highly effective tool for cancer ablation without the use of ionizing radiation and with minimal off-target toxicity. See Cengel et al., 2016. The mechanism of PDT relies on the generation of reactive oxygen species (ROS) with a combination of a photosensitizer, light, and oxygen. See Wilson and Patterson, 2008. Due to their extremely short lifetime, ROS can diffuse only several nanometers in tissue, which confines PDT cytotoxic action to the area irradiated by light. Accordingly, PDT is highly advantageous in cases when it is crucial to preserve the function of the surrounding healthy tissues. Plaetzer et al., 2009. PDT can readily be combined with various imaging modalities, such as nuclear imaging or fluorescence imaging, for simultaneous tumor detection and image-guided therapy. See Celli et al., 2010; Mallidi et al., 2016.

With the advent of endoscopic light delivery technologies, PDT became available for a variety of deep-seated tumors via minimally-invasive intracavitary or interstitial approaches. Abrahamse and Hamblin, 2016. For example, vascular-targeted PDT with a water-soluble photosensitizer, padeliporfin, has been investigated for low-risk, localized prostate cancer treatment. See Trachtenberg et al., 2007; Taneja et al., 2016; and Trachtenberg et al., 2008. In that approach, patients were administered photosensitizer intravenously and the prostate was irradiated by interstitially positioned optical fibers, with photosensitizer within the vascular compartment. That led to vascular occlusion, resulting in tumor regression. A recently completed multicenter phase III randomized trial demonstrated that vascular targeted PDT decreased the percentage of patients with progressive disease from 58% to 28% and increased the number of patients with disease-free biopsy from 28% to 49% when compared to those receiving the standard-of-care. See Azzourzi et al., 2017. Overall, the results suggest that PDT holds significant promise for managing cancer, such as prostate cancer.

### SUMMARY

The presently disclosed subject matter provides a long-circulating PSMA-targeted phototheranostic agent for multimodal imaging and cancer therapy.

In some aspects, the presently disclosed subject matter provides a theranostic probe comprising a porphyrin-based photosensitizer capable of multimodal positron emission tomography (PET)/fluorescence imaging and photodynamic therapy; a peptide linker to impart water solubility and to prolong circulation time; and a urea-based high-affinity PSMA-targeting ligand.

In certain aspects, the theranostic probe comprises a compound of formula (Ia):

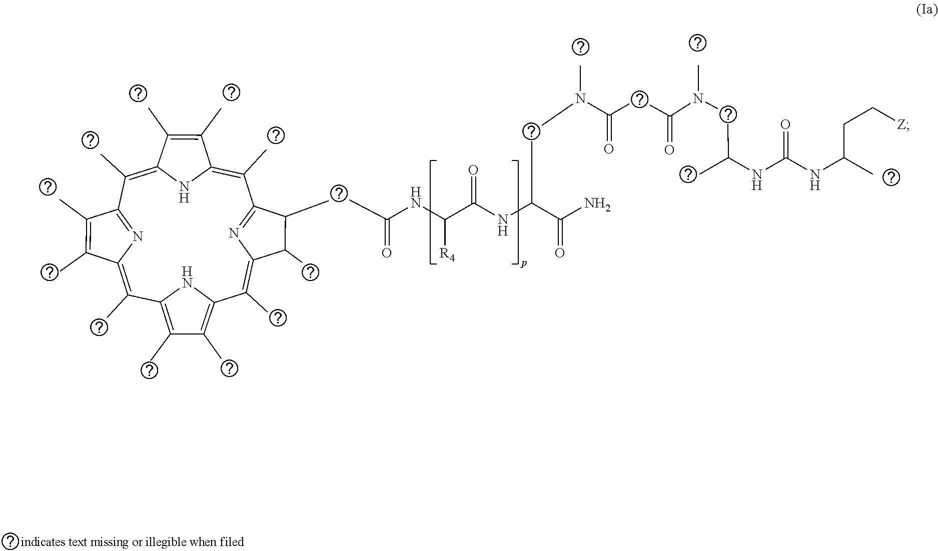

wherein: m1 and m2 are each independently an integer selected from the group consisting of 1, 2, 3, 4, 5, 6, 7, and 8;

n1 and n2 are each independently an integer selected from the group consisting of 1, 2, 3, 4, 5, 6, 7, and 8;

p is an integer selected from the group consisting of 5, 6, 7, 8, 9, 10, 11, 12, 13, 14, and 15;

Z is tetrazole or —CO₂Q;

each Q is independently selected from the group consisting of hydrogen, substituted or unsubstituted straight-chain or branched C₁-C₈ alkyl, substituted or unsubstituted aryl, and a protecting group; and

R₁ and R₂ are each independently selected from the group consisting of hydrogen; substituted or unsubstituted straight-chain or branched alkyl, substituted or unsubstituted aryl;

R₃ₐ, R_{3b}, R_{3c}, R_{3d}, R₃ₑ, R_{3f}, R_{3g}, R₃ₕ, R₃ᵢ, R₃ⱼ, and R₃ₖ are each independently selected from the group consisting of substituted or unsubstituted straight-chain or branched C₁-C₈ alkyl, substituted or unsubstituted C₁-C₈ alkenyl, substituted or unsubstituted aryl, wherein the aryl can be substituted with one or more substituent groups selected from substituted or unsubstituted straight-chain or branched C₁-C₈ alkyl, hydroxyl, C₁-C₈ alkoxyl, amino, cyano, carboxyl, halogen, —SO₃⁻, and oxo;

or R₃ₐ and R_{3b}, R_{3c} and R_{3d}, R_{3d} and R₃ₑ, R_{3f} and R_{3g}, R_{3g} and R₃ₕ, R₃ᵢ and R₃ⱼ, and R₃ⱼ and R₃ₖ can together form a 5- to 6-member carbocyclic ring along with the porphyrin ring, which can be substituted with one or more substituent groups selected from substituted or unsubstituted straight-chain or branched C₁-C₈ alkyl, hydroxyl, C₁-C₈ alkoxyl, amino, cyano, carboxyl, halogen, and oxo; and

each R₄ is independently selected from the group consisting of substituted or unsubstituted straight-chain or branched C₁-C₈ alkyl, substituted or unsubstituted C₁-C₈ alkenyl, substituted or unsubstituted aryl, —(CH₂)ₙ₃—OR₅, —(CH₂)ₙ₄—CO₂R₆, —NR₇R₈, —SR₉, —SeR₁₀, substituted or unsubstituted cycloheteroalkyl, substituted or unsubstituted aryl, substituted or unsubstituted arylalkyl, substituted or unsubstituted heteroarylalkyl, and substituted or unsubstituted heteroaryl;

wherein n3 and n4 are each independently an integer selected from the group consisting of 1, 2, 3, 4, 5, 6, 7, and 8; and

R₅, R₆, R₇, R₅, R₉, and R₁₀ are each independently selected from the group consisting of hydrogen and substituted or unsubstituted straight-chain or branched C₁-C₈ alkyl; and

pharmaceutically acceptable salts thereof.

In more certain aspects, the theranostic probe has the following chemical structure:

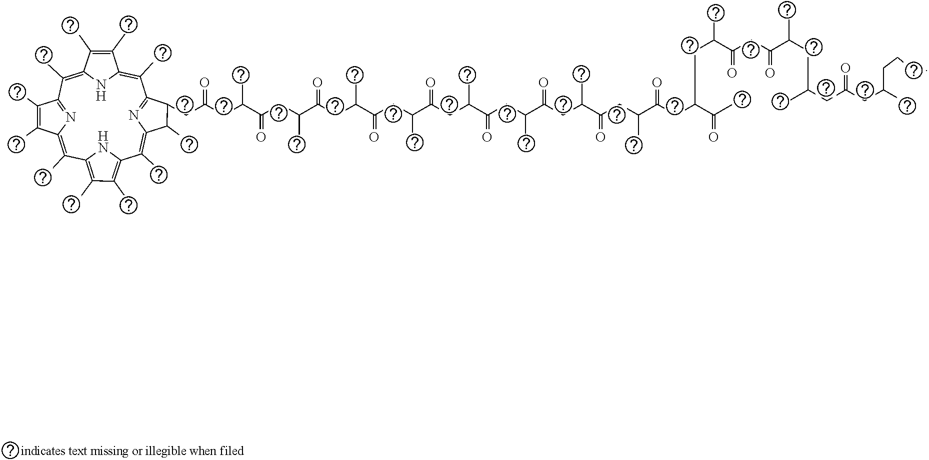 [SMILES: CCCC(C)CC(=O)CC(C)CC(C)C(=O)CC(=O)C(C)CC(CC(=O)C(C)CC(=O)C(C)CC(=O)C(C)CC(=O)C(C)CC(=O)C(C)CC(=O)C(C)CC(=O)C(C)CC(=O)C(C)CC(=O)CC1c2nc(c(C)c3[nH]c(c(C)c4nc(c(C)c5[nH]c(c2C)c(C)c5C)C(C)=C4C)c(C)c3C)C1C)C(C)=O]

In yet more certain aspects, the theranostic probe has the following chemical structure:

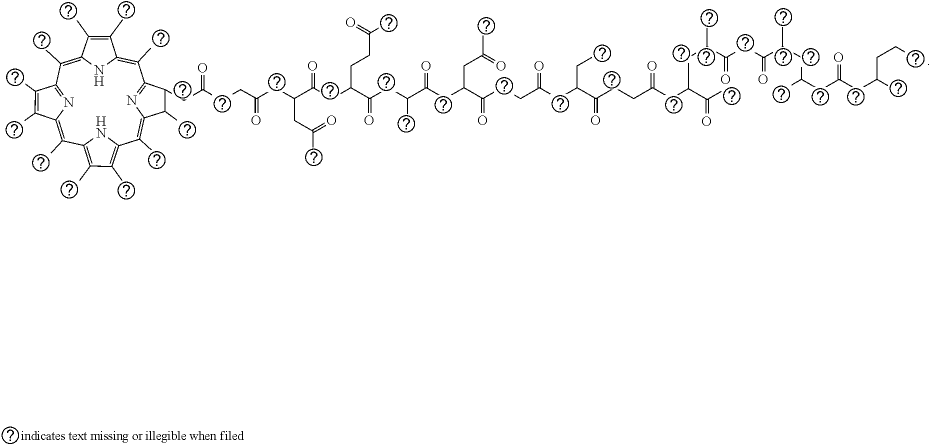 [SMILES: CCCC(C)CC(=O)CC(C)CC(C)C(=O)CC(=O)C(C)CC(CC(=O)CCC(=O)C(CC)CC(=O)CCC(=O)C(CC(C)=O)CC(=O)C(C)CC(=O)C(CCC(C)=O)CC(=O)C(CC(C)=O)CC(=O)CCC(=O)CC1c2nc(c(C)c3[nH]c(c(C)c4nc(c(C)c5[nH]c(c2C)c(C)c5C)C(C)=C4C)c(C)c3C)C1C)C(C)=O]

In even yet more certain aspects, the theranostic probe has the following chemical structure:

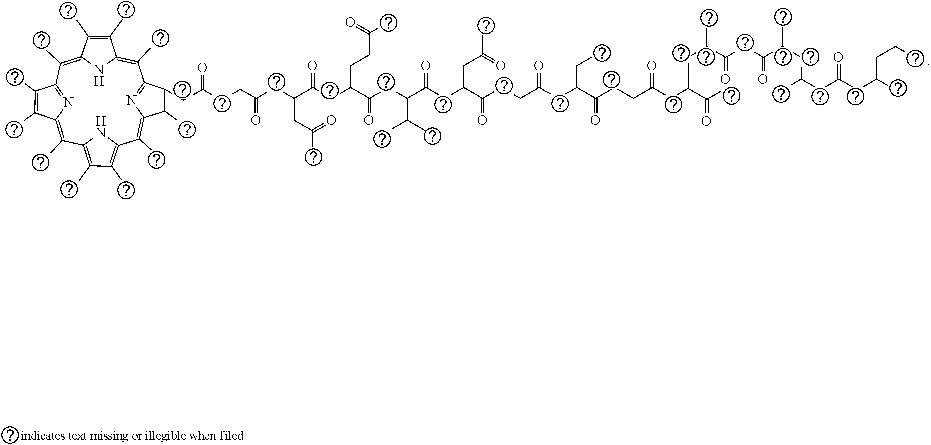 [SMILES: CCCC(C)CC(=O)CC(C)CC(C)C(=O)CC(=O)C(C)CC(CC(=O)CCC(=O)C(CC)CC(=O)CCC(=O)C(CC(C)=O)CC(=O)C(CC(=O)C(CCC(C)=O)CC(=O)C(CC(C)=O)CC(=O)CCC(=O)CC1c2nc(c(C)c3[nH]c(c(C)c4nc(c(C)c5[nH]c(c2C)c(C)c5C)C(C)=C4C)c(C)c3C)C1C)C(C)C)C(C)=O]

In particular aspects, the theranostic probe has the following chemical structure:

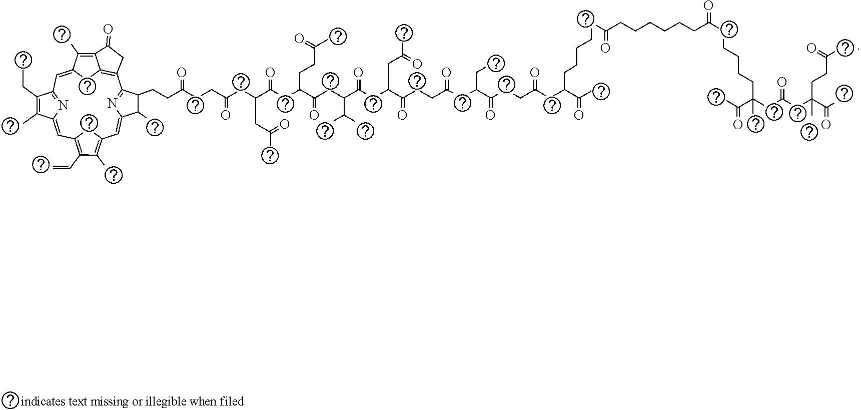 [SMILES: C=CC1=C2/C=C3N=C(/C=C4\CC5=C(CC(=O)C5=C4C)C4=N/C(=C\C(=C1C)C2)C(C)C4CCC(=O)CCC(=O)CC(CC(C)=O)C(=O)CC(CCC(C)=O)C(=O)CC(C(=O)CC(CC(C)=O)C(=O)CCC(=O)CC(CC)C(=O)CCC(=O)CC(CCCCCC(=O)CCCCCCC(=O)CCCCCC(C)(CC(=O)CC(C)(CCC(C)=O)C(C)=O)C(C)=O)C(C)=O)C(C)C)C(CC)=C\3C]

In some aspects, the photosensitizer further comprises a radiometal. In particular aspects, the radiometal has a t_{1/2} greater than about three hours. In yet more particular aspects, the radiometal is selected from the group consisting of ⁶⁴Cu, ⁶¹Cu, ⁶⁷Cu, ¹¹¹In, ⁸⁹Zr, and ⁶⁸Ga.

In other aspects, the presently disclosed subject matter provides a method for treating or imaging one or more PSMA expressing tumors or cells, the method comprising contacting the one or more PSMA expressing tumors or cells with an effective amount of the presently disclosed theranostic probe.

In particular aspects, the prostate-specific membrane antigen (PSMA)-positive tumor or cell is selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof.

In certain aspects, the presently disclosed method further comprises taking an image. In yet more certain aspects, the taking of an image comprises positron emission tomography (PET).

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 is a schematic diagram of the presently disclosed theranostic probe, referred to herein as LC-Pyro (long-circulating pyropheophorbide α), which is comprised of three building blocks: (1) a porphyrin photosensitizer capable of deep-red fluorescence imaging and ⁶⁴Cu-chelated PET imaging; (2) a 9-amino acid D-peptide sequence that imparts water-solubility and prolongs plasma circulation to promote tumor accumulation; and (3) a high-affinity urea-based small-molecule PSMA targeting ligand;

FIG. 2A, FIG. 2B, FIG. 2C, and FIG. 2D show structures of PSMA conjugates and the photonic properties of LC-Pyro. FIG. 2A shows structures of LC-Pyro (Long-circulating pyropheophorbide α), SC-Pyro (Short-circulating pyropheophorbide α) and DCIBzL; FIG. 2B shows a LC-Pyro absorbance spectrum; FIG. 2C is a fluorescence emission spectrum; and FIG. 2D shows reactive oxygen species generation from LC-Pyro in solution with respect to 671-nm laser light dose (n=3 from three independent measurements);

FIG. 3A, FIG. 3B, FIG. 3C, FIG. 3D, and FIG. 3E show PSMA targeting selectivity and specificity in vitro. FIG. 3A shows selectivity of LC-FITC uptake in PSMA+ PC3 PIP and PSMA− PC3 flu cells; FIG. 3B shows specificity of PSMA targeting ligand with LC-FITC uptake in PSMA+ PC3 PIP cells in the presence of 10-, 50- and 100-fold excess DCIBzL for target blockade; FIG. 3C shows representative flow cytometry histogram plots with time-dependent quantification of LC-FITC uptake in PSMA− PC3-flu (open black square) and PSMA+ PC3-PIP (solid green circle) cells; FIG. 3D shows representative fluorescence micrographs of time-dependent PSMA+ PC3-PIP cell uptake of LC-FITC up to 6 hours incubation; FIG. 3E is a Western blot of PC3, PSMA− PC3 flu, PSMA+ PC3 PIP, PC3-ML-1117 and PC3-ML-1124 cell lysates using anti-PSMA and β-actin antibodies. Scale=20 μm. Each point represents the median±SD of three independent measurements;

FIG. 4A, FIG. 4B, FIG. 4C, FIG. 4D, FIG. 4E, FIG. 4F, and FIG. 4G demonstrate the pharmacokinetic role of the peptide linker in LC-Pyro for its ability to accumulate in PSMA-expressing tumors; FIG. 4A shows blood clearance curves of LC-Pyro and SC-Pyro (n=5 each group) in BALB/c mice with blood samples collected over a 48-hour period. The profiles fit into a two-compartment model with a half-life of 10.00 hours for LC-Pyro and 1.17 hours for SC-Pyro; FIG. 4B is a table including the Ki inhibitory activities of SC-Pyro, LC-Pyro and DCIBzL against PSMA determined using a fluorescence-based assay; FIG. 4C and FIG. 4D are representative fluorescence images of mice bearing dual PSMA+ PC3 PIP (red arrow) and PSMA− PC3 flu (green arrow) tumors that were intravenously injected either with LC-Pyro (FIG. 4C) or SC-Pyro (FIG. 4D) at 0.5, 1, 6 and 24 hours post-injection; FIG. 4E and FIG. 4F show the fluorescence ex vivo organ distribution of mice injected with LC-Pyro (FIG. 4E) or SC-Pyro (FIG. 4F) including PSMA+ PC3 PIP tumor (insets); FIG. 4G shows PSMA inhibition in vivo with 150× molar excess of DCIBzL intravenously injected 30 minutes prior to LC-Pyro intravenous injection. Mice were sacrificed 24 hours post-injection and tumors were excised for ex vivo fluorescence comparison. All images displayed are comparable with the same integration time;

FIG. 5A, FIG. 5B, and FIG. 5C show ⁶⁴Cu-LC-Pyro-enabled PET imaging in an orthotopic prostate cancer model and fluorescence detection of PSMA+ micrometastases with LC-Pyro. FIG. 5A shows representative sagittal PET/CT in PSMA+ PC3 PIP and PSMA-PC3 flu orthotopic prostate cancer mice at 3 hours and 17 hours after intravenous administration of ⁶⁴Cu-LC-Pyro; FIG. 5B show ⁶⁴Cu-labeled LC-Pyro biodistribution in the tumors and the surrounding organs quantified via gamma counting (n=4 for PSMA+ PC3 PIP; n=3 for PSMA− PC3 flu, **P<0.01; **P<0.001); and FIG. 5C shows in situ bioluminescence images of mice bearing PSMA+ (PC3-ML-1124) and PSMA− (PC3-ML-1117) metastatic nodules (internal organs removed, to expose retroperitoneal cavity). Corresponding fluorescence images demonstrating specific uptake of LC-Pyro in the PSMA+ nodule and the fluorescence microscopic analysis of 10 μm frozen sections (LC-Pyro-red; DAPI-blue; scale=20 μm); and

FIG. 6A, FIG. 6B, FIG. 6C, and FIG. 6D demonstrate the PDT efficacy of LC-Pyro in PSMA+ PC3 PIP tumor-bearing mice. FIG. 6A shows tumor growth curves represented as average tumor volume±standard deviation (n=4 for each group); FIG. 6B shows body mass curves (average±standard deviation); FIG. 6C shows representative images of tumor-burdened mice in four treatment groups: 1) saline only, 2) laser only, 3) LC-Pyro only and 4) LC-Pyro+Laser at 0, 6 and 22 days post-PDT treatment; and FIG. 6D shows H&E and TUNEL staining of tumor sections from saline only, laser only, LC-Pyro only and LC-Pyro+Laser groups at 24 hours post-treatment; scale=50 μm;

FIG. 7 shows chemical characterization of Pyro-peptide, LC-Pyro and SC-Pyro; uPLC retention profile (Top); Corresponding UV-vis absorbance spectra (Middle) and mass spectra (Bottom);

FIG. 8 shows flow cytometry time-dependent LC-Pyro uptake in PSMA+ PC3 PIP and PSMA− PC3 flu cell lines. Each point represents the median fluorescence intensity±the SD of three independent measurements;

FIG. 9 shows representative whole-body fluorescence images of a mouse bearing dual PSMA+ PC3 PIP (red arrow) and PSMA− PC3 flu (green arrow) tumors that were intravenously injected with SC-Pyro (20 nmol) at 0, 15 min, 30 min, 1 h, 6 h and 24 h post-injection; n=3;

FIG. 10 shows fluorescence ex vivo biodistribution of major clearance organs in a mouse injected with LC-FITC 24 h post-injection;

FIG. 11 shows representative whole-body fluorescence images of a mouse bearing dual PSMA+ PC3 PIP (red arrow) and PSMA− PC3 flu (green arrow) tumors that were intravenously injected with an LC-Pyro derivative unconjugated to the PSMA small molecule affinity ligand (20 nmol). Mice were imaged at 0, 30 min, 1 h, 6 h and 24 h post-intravenous injection and major organs were excised and imaged ex vivo; n=3;

FIG. 12 shows lateral view PET/CT images of mice bearing either a PSMA− PC3 flu (left) or PSMA+ PC3 PIP (right) orthotopic prostate tumor 17 hours post-injection of ⁶⁴Cu-LC-Pyro;

FIG. 13 shows haemotoxylin and eosin (H&E) stained sections for evaluation of organ toxicity 24 hours post-PDT treatment from each cohort: (1) Saline only; (2) Laser only; (3) LC-Pyro only; and (4) LC-Pyro+Laser. Organs include the liver, lung, skin, small intestine, muscle, adrenal, large intestine, kidney, heart and spleen. Histological slices reveal no adverse side effects on healthy tissues after treatment. Scale=50 μm;

FIG. 14 shows representative PSMA staining sections of PSMA+ PC3 PIP tumors post-PDT treatment from each cohort for immunohistochemical validation of PSMA expression: (1) Saline only; (2) Laser only; (3) LC-Pyro only; and (4) LC-Pyro+Laser. Scale=50 μm; and

FIG. 15 shows the chemical structure of reported compound YC-9 (Chang et al., 1999 (prior art)) and its plasma blood clearance profile in BALB/c mice (n=5). The profile fits into a two-compartment model with a slow half-life of 0.60 hours.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Long-circulating PSMA-Targeted Phototheranostic Agent

A. Theranostic probes of Formula (I)

In some embodiments, the presently disclosed subject matter provides a theranostic probe comprising a compound of formula (I):

P-L-T (I)

wherein:

P is a porphyrin-based photosensitizer;

L is a peptide linker; and

T is a urea-based PSMA-targeting ligand; and

pharmaceutically acceptable salts thereof.

In particular embodiments of the presently disclosed theranostic probe, the photosensitizer comprises a porphyrin-based photosensitizer. As used herein, porphyrin refers to a heterocyclic macrocyclic organic compound comprising four modified pyrrole subunits interconnected at their α-carbon atoms via methane bridges. Porphyrins comprise the following core structure, which can be substituted with one or more substituent groups, generally shown immediately herein as “R”:

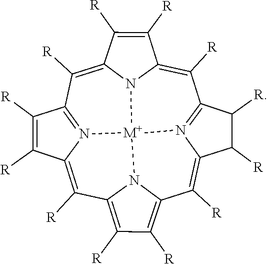

Sidechains including, but not limited to, functional groups comprising nitrogen-containing groups, carboxylic acids, and sugars can be incorporated into the porphyrin core structure, such as diethylaminopentyl sidechains, phenyl groups, phenoxyl groups, pyrrolidinyl, isoquinoline moieties, silyl groups, and the like. See Zhang et al., Acta Pharmaceutica Sinica B, 2017. Other tetrapyrrole-type photosensitizers, including, but not limited to, chlorins, bacteriochlorins, and phthalocyanines, see Abrahamse and Hamblin, Biochem. J. 2016, also are suitable for use with the presently disclosed methods. In other embodiments, the photosensitizer is TOOKAD® (Steba Biotech, Luxembourg), which has the following formula:

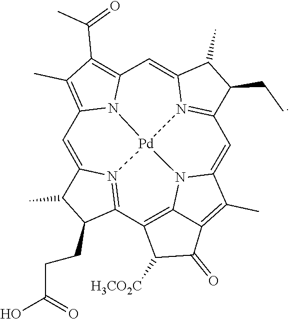

In particular embodiments, the photosensitizer is pyropheophorbide α:

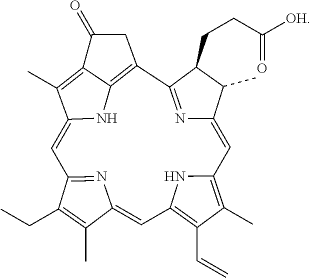 [SMILES: C=Cc1c(C)c2cc3nc(c4c5[nH]c(cc6nc(cc1[nH]2)C(C)=C6CC)c(C)c5C(=O)C4)[C@@H](CCC(=O)O)[C@@H]3C]

Porphyrins can form complexes with a metal, M+. In certain embodiments, the metal can have a M⁺¹, M⁺², or M³ charge. In yet more certain embodiments of the presently disclosed subject matter, the metal is a radiometal suitable for use with positron emission tomography (PET).

In certain embodiments, the photosensitizer further comprises a radiometal. In more certain embodiments, the radiometal has a t_{1/2} greater than about three hours. In some embodiments, the t_{1/2} is greater than about three hours. In some embodiments, the t_{1/2} is greater than about 3.5 hours. In some embodiments, the t_{1/2} is greater than about four hours. In some embodiments, the t_{1/2} is greater than about 4.5 hours. In some embodiments, the t_{1/2} is greater than about five hours. In some embodiments, the t_{1/2} is greater than about 3 hrs, about 3.5 hrs, about 4 hrs, about 4.5 hrs, about 5 hrs, about 5.5 hrs, about 6 hrs, about 6.5 hrs, about 7 hrs, about 7.5 hrs, about 8 hrs, about 8.5 hrs, about 9 hrs, about 9.5 hrs, about 10 hrs, about 10.5 hrs, about 11 hrs, about 11.5 hrs, about 12 hrs, about 12.5 hrs, about 13 hrs, about 13.5 hrs, about 14 hrs, about 14.5 hrs, about 15 hrs, and beyond. In yet more certain embodiments, the radiometal is selected from the group consisting of ⁶⁴Cu, ⁶¹Cu, ⁶⁷Cu, ¹¹¹In, ⁸⁹Zr, and ⁶⁸Ga. In yet more certain embodiments, the photosensitizer is capable of multimodal fluorescence imaging and radioimaging. In even yet more certain embodiments, the radioimaging is positron emission tomography (PET) imaging. In other embodiments, the radioimaging is single photon computed emission tomography (SPECT) imaging.

In some embodiments, the peptide linker comprises a D-peptide sequence comprising from about 5 to about 15 D-amino acids. In particular embodiments, the D-peptide sequence comprises nine amino acids. In yet more particular embodiments, the D-peptide sequence is GDEVDGSGK, which is disclosed in U.S. Pat. No. 8,133,482 to Zheng et al., issued March 13, 2012, which is incorporated herein by reference in its entirety. In other embodiments, the D-peptide sequence is FAEKFKEAVKDYFAKFWD.

As used herein, the term “amino acid” includes moieties having a carboxylic acid group and an amino group. The term amino acid thus includes both natural amino acids (including proteinogenic amino acids) and non-natural amino acids. The term “natural amino acid” also includes other amino acids that can be incorporated intoproteins during translation (including pyrrolysine and selenocysteine). Additionally, the term“natural amino acid” also includes other amino acids, which are formed during intermediary metabolism, e.g., ornithine generated from arginine in the urea cycle. The natural amino acids are summarized below in Table 1:

TABLE 1
Natural Amino Acids

| Amino Acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |

The natural or non-natural amino acid may be optionally substituted. In one embodiment, the amino acid is selected from proteinogenic amino acids.

Proteinogenic amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine. The term amino acid includes alpha amino acids and beta amino acids, such as, but not limited to, beta alanine and 2-methyl beta alanine. The term amino acid also includes certain lactam analogues of natural amino acids, such as, but not limited to, pyroglutamine. The term amino acid also includes amino acids homologues including homocitrulline, homoarginine, homoserine, homotyrosine, homoproline and homophenylalanine.

The terminal portion of the amino acid residue or peptide may be in the form of the free acid i.e., terminating in a —COOH group or may be in a masked (protected) form, such as in the form of a carboxylate ester or carboxamide. In certain embodiments, the amino acid or peptide residue terminates with an amino group. In an embodiment, the residue terminates with a carboxylic acid group —COOH or an amino group —NH₂. In another embodiment, the residue terminates with a carboxamide group. In yet another embodiment, the residue terminates with a carboxylate ester.

As disclosed hereinabove, the term “amino acid” includes compounds having a —COOH group and an —NH₂ group. A substituted amino acid includes an amino acid which has an amino group which is mono- or di-substituted. In particular embodiments, the amino group may be mono-substituted. (A proteinogenic amino acid may be substituted at another site from its amino group to form an amino acid which is a substituted proteinogenic amino acid). The term substituted amino acid thus includes N-substituted metabolites of the natural amino acids including, but not limited to, N-acetyl cysteine, N-acetyl serine, and N-acetyl threonine.

For example, the term“N-substituted amino acid” includes N-alkyl amino acids (e.g., C₁-C₆ N-alkyl amino acids, such as sarcosine, N-methyl-alanine, N-methyl-glutamic acid and N-tert-butylglycine), which can include C₁-C₆ N-substituted alkyl amino acids (e.g., N-(carboxy alkyl) amino acids (e.g., N-(carboxymethyl)amino acids) and N-methylcycloalkyl amino acids (e.g., N-methylcyclopropyl amino acids)); N,N-di-alkyl amino acids (e.g., N,N-di-C₁-C₆ alkyl amino acids (e.g., N,N-dimethyl amino acid)); N,N,N-tri-alkyl amino acids (e.g., N,N,N-tri-C₁-C₆ alkyl amino acids (e.g., N,N,N-trimethyl amino acid)); N-acyl amino acids (e.g., C₁-C₆ N-acyl amino acid); N-aryl amino acids (e.g., N-phenyl amino acids, such as N-phenylglycine); N-amidinyl amino acids (e.g., an N-amidine amino acid, i.e., an amino acid in which an amine group is replaced by a guanidino group).

The term“amino acid” also includes amino acid alkyl esters (e.g., amino acid C₁-C₆ alkyl esters); and amino acid aryl esters (e.g., amino acid phenyl esters).

For amino acids having a hydroxy group present on the side chain, the term “amino acid” also includes O-alkyl amino acids (e.g., C₁-C₆ O-alkyl amino acid ethers); O-aryl amino acids (e.g., O-phenyl amino acid ethers); O-acyl amino acid esters; and O-carbamoyl amino acids.

For amino acids having a thiol group present on the side chain, the term “amino acid” also includes S-alkyl amino acids (e.g., C₁-C₆ S-alkyl amino acids, such as S-methyl methionine, which can include C₁-C₆ S-substituted alkyl amino acids and S-methylcycloalkyl amino acids (e.g., S-methylcyclopropyl amino acids)); S-acyl amino acids (e.g., a C₁-C₆ S-acyl amino acid); 5-aryl amino acid (e.g., a S-phenyl amino acid); a sulfoxide analogue of a sulfur-containing amino acid (e.g., methionine sulfoxide) or a sulfoxide analogue of an S-alkyl amino acid (e.g., S-methyl cystein sulfoxide) or an S-aryl amino acid.

In other words, the presently disclosed subject matter also envisages derivatives of natural amino acids, such as those mentioned above which have been functionalized by simple synthetic transformations known in the art (e.g., as described in“Protective Groups in Organic Synthesis” by T W Greene and P G M Wuts, John Wiley & Sons Inc. (1999)), and references therein.

Examples of non-proteinogenic amino acids include, but are not limited to: citrulline, hydroxyproline, 4-hydroxyproline, β-hydroxyvaline, ornithine, β-amino alanine, albizziin, 4-amino-phenylalanine, biphenylalanine, 4-nitro-phenylalanine, 4-fluoro-phenylalanine, 2,3,4,5,6-pentafluoro-phenylalanine, norleucine, cyclohexylalanine, α-aminoisobutyric acid, α-aminobutyric acid, α-aminoisobutyric acid, 2-aminoisobutyric acid, 2-aminoindane-2-carboxylic acid, selenomethionine, lanthionine, dehydroalanine, γ-amino butyric acid, naphthylalanine, aminohexanoic acid, pipecolic acid, 2,3-diaminoproprionic acid, tetrahydroisoquinoline-3-carboxylic acid, tert-leucine, tert-butylalanine, cyclopropylglycine, cyclohexylglycine, 4-aminopiperidine-4-carboxylic acid, diethylglycine, dipropylglycine and derivatives thereof wherein the amine nitrogen has been mono- or di-alkylated.

The term“peptide” refers to an amino acid chain consisting of 2 to 50 amino acids, unless otherwise specified. More particularly, the term D-peptide refers to a small sequence of D-amino acids. In preferred embodiments, the peptide used in the present invention is about 5 to about 15 amino acids in length. In particular embodiments, the peptide comprises nine amino acids. In yet more particular embodiments, the peptide comprises GDEVDGSGK. In one embodiment, the peptide can be a branched peptide. In this embodiment, at least one amino acid side chain in the peptide is bound to another amino acid (either through one of the termini or the side chain).

The term “N-substituted peptide” refers to an amino acid chain consisting of 2 to 50 amino acids in which one or more NH groups are substituted, e.g., by a substituent described elsewhere herein in relation to substituted amino groups.

Optionally, the N-substituted peptide has its N-terminal amino group substituted and, in one embodiment, the amide linkages are unsubstituted.

In one embodiment, an amino acid side chain is bound to another amino acid. In a further embodiment, side chain is bound to the amino acid via the amino acid's N-terminus, C-terminus, or side chain.

Examples of natural amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), —CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

In some embodiments, the urea-based PSMA-targeting ligand comprises the following chemical moiety:

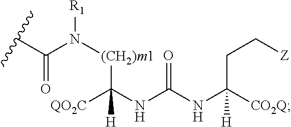

wherein:

m1 is an integer selected from 1, 2, 3, 4, 5, 6, 7, and 8;

Z is tetrazole or —CO₂Q;

each Q is independently selected from the group consisting of hydrogen, substituted or unsubstituted straight-chain or branched C₁-C₈ alkyl, substituted or unsubstituted aryl, and a protecting group; and

R₁ is selected from the group consisting of hydrogen, substituted or unsubstituted straight-chain or branched C₁-C₈ alkyl, and substituted or unsubstituted aryl.

In certain embodiments, the theranostic probe comprises a compound of formula (Ia):

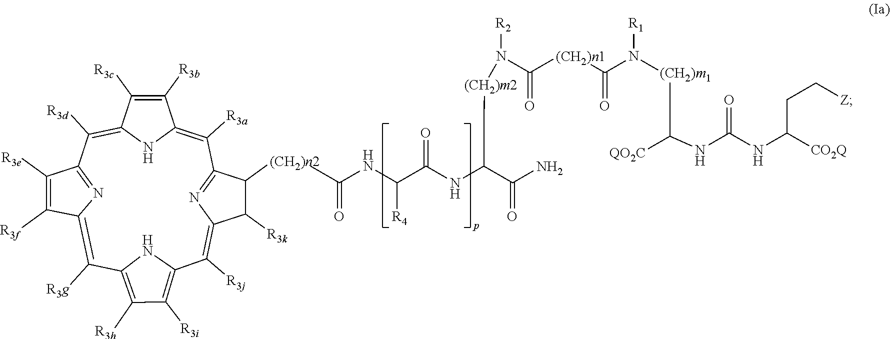

wherein:

m1 and m2 are each independently an integer selected from the group consisting of 1, 2, 3, 4, 5, 6, 7, and 8;

n1 and n2 are each independently an integer selected from the group consisting of 1, 2, 3, 4, 5, 6, 7, and 8;

p is an integer selected from the group consisting of 5, 6, 7, 8, 9, 10, 11, 12, 13, 14, and 15;

Z is tetrazole or —CO₂Q;

each Q is independently selected from the group consisting of hydrogen, substituted or unsubstituted straight-chain or branched C₁-C₈ alkyl, substituted or unsubstituted aryl, and a protecting group; and

R₁ and R₂ are each independently selected from the group consisting of hydrogen; substituted or unsubstituted straight-chain or branched alkyl, substituted or unsubstituted aryl;

R₃ₐ, R_{3b}, R_{3c}, R_{3d}, R₃ₑ, R_{3f}, R_{3g}, R₃ₕ, R₃ᵢ, R₃ⱼ, and R₃ₖ are each independently selected from the group consisting of substituted or unsubstituted straight-chain or branched C₁-C₈ alkyl, substituted or unsubstituted C₁-C₈ alkenyl, substituted or unsubstituted aryl, wherein the aryl can be substituted with one or more substituent groups selected from substituted or unsubstituted straight-chain or branched C₁-C₈ alkyl, hydroxyl, C₁-C₈ alkoxyl, amino, cyano, carboxyl, halogen, —SO₃⁻, and oxo;

or R₃ₐ and R_{3b}, R_{3c} and R_{3d}, R_{3d} and R₃ₑ, R_{3f} and R_{3g}, R_{3g} and R₃ₕ, R₃i and R₃ⱼ, and R₃ⱼ and R₃ₖ can together form a 5- to 6-member carbocyclic ring along with the porphyrin ring, which can be substituted with one or more substituent groups selected from substituted or unsubstituted straight-chain or branched C₁-C₈ alkyl, hydroxyl, C₁-C₈ alkoxyl, amino, cyano, carboxyl, halogen, and oxo; and
each R₄ is independently selected from the group consisting of substituted or unsubstituted straight-chain or branched C₁-C₈ alkyl, substituted or unsubstituted C₁-C₈ alkenyl, substituted or unsubstituted aryl, —(CH₂)ₙ₃—OR₅, —(CH₂)ₙ₄—CO₂R₆, —NR₇R₈, —SR₉, —SeR₁₀, substituted or unsubstituted cycloheteroalkyl, substituted or unsubstituted aryl, substituted or unsubstituted arylalkyl, substituted or unsubstituted heteroarylalkyl, and substituted or unsubstituted heteroaryl;

wherein n3 and n4 are each independently an integer selected from the group consisting of 1, 2, 3, 4, 5, 6, 7, and 8; and

R₅, R₆, R₇, R₈, R₉, and R₁₀ are each independently selected from the group consisting of hydrogen and substituted or unsubstituted straight-chain or branched C₁-C₈ alkyl; and

pharmaceutically acceptable salts thereof.

In more certain embodiments, the theranostic probe has the following chemical structure:

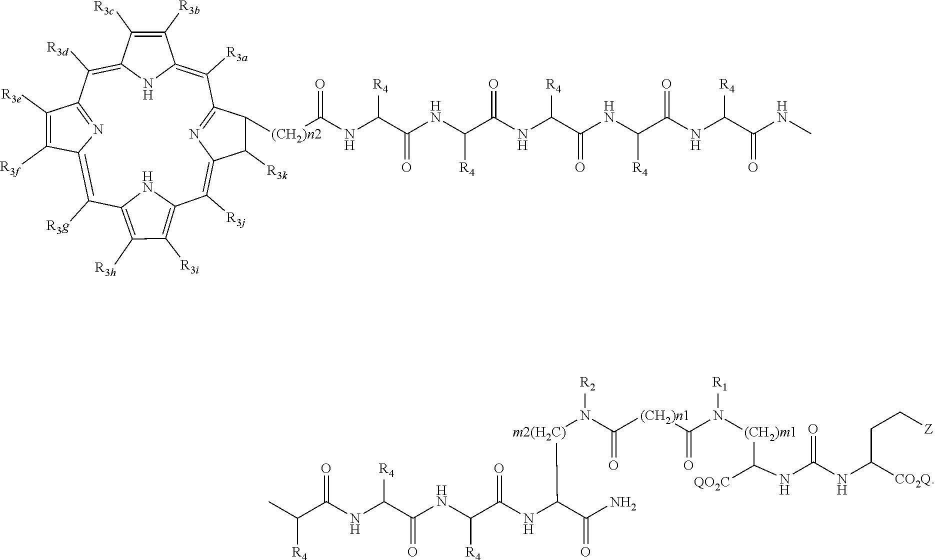

In yet more certain embodiments, the theranostic probe has the following chemical structure:

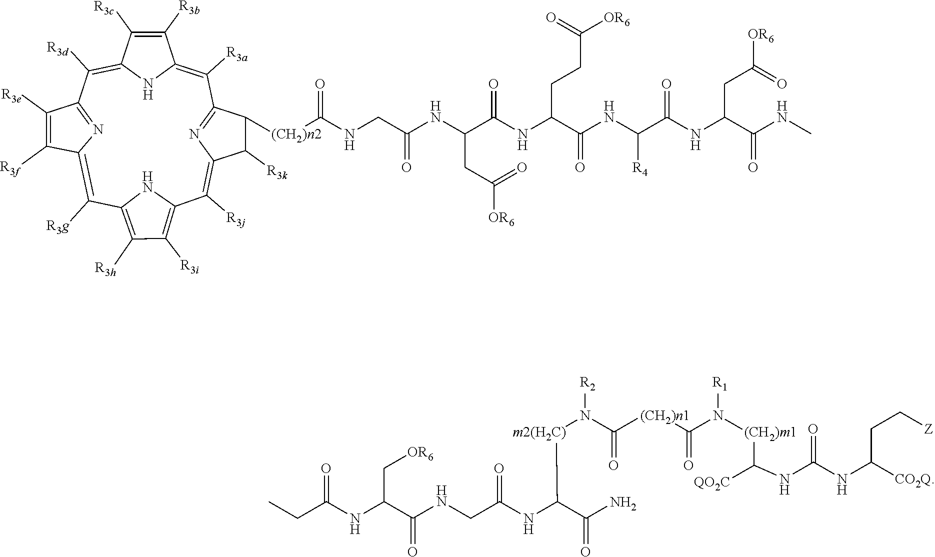

In even yet more certain embodiments, the theranostic probe has the following chemical structure:

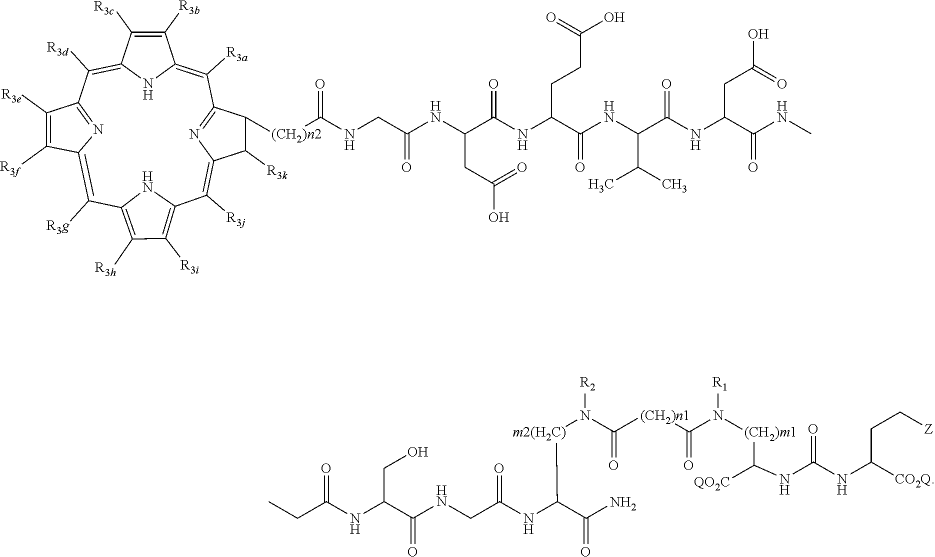

In particular embodiments, the theranostic probe has the following chemical structure:

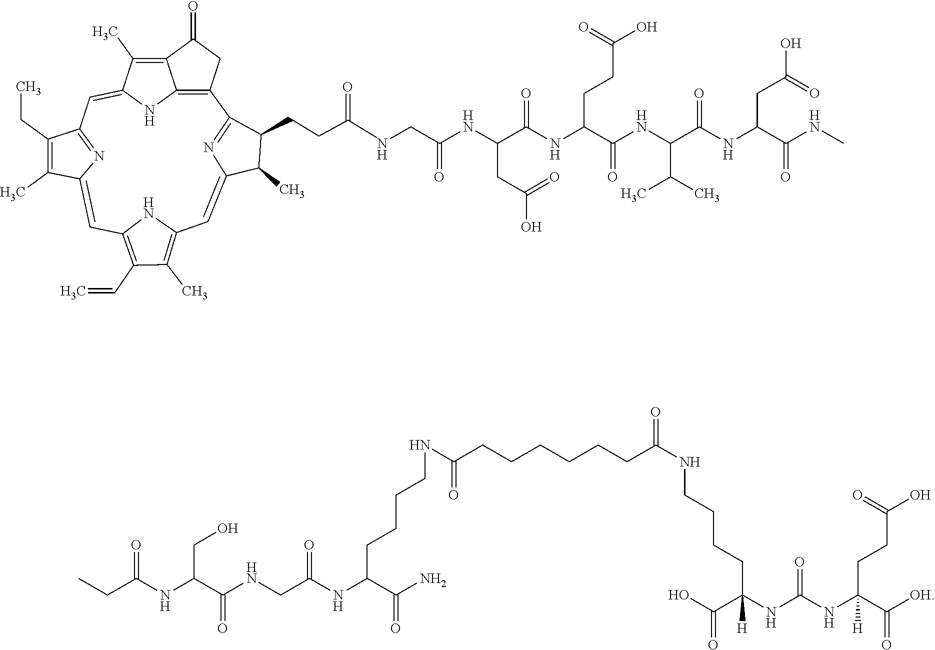

In some embodiments, the photosensitizer further comprises a radiometal. In some embodiments, the t_{1/2} is greater than about three hours. In particular embodiments, the radiometal is selected from the group consisting of ⁶⁴Cu, ⁶¹Cu, ⁶⁷Cu,¹¹¹In, ⁸⁹Zr, and ⁶⁸Ga.

B. Methods for Imaging a Prostate-Specific Membrane Antigen (PSMA)-Positive Tumor or Treating a Disease, Disorder, or Condition Associated with PSMA

In other embodiments, the presently disclosed subject matter provides a method for treating or imaging one or more PSMA expressing tumors or cells, the method comprising contacting the one or more PSMA expressing tumors or cells with an effective amount of the presently disclosed theranostic probe.

In some embodiments or the presently disclosed methods, the prostate-specific membrane antigen (PSMA)-positive tumor or cell is selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof.

In some embodiments, the presently disclosed theranostic probe extends plasma circulation time up to 10 hours, including 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0 hours, compared to a truncated derivative having a linker comprising a lysine residue only. This characteristic also increases tumor accumulation.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

In other embodiments, the presently disclosed method further comprises taking an image. In particular embodiments, the taking of an image comprises positron emission tomography (PET).

In other embodiments, the one or more PSMA-expressing tumors or cells is in vitro, in vivo or ex-vivo. In yet other embodiments, the one or more PSMA-expressing tumor or cell is present in a subject.

In general, the “effective amount” of an active agent refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a presently disclosed theranostic probe and at least one other active agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

C. Kits

In certain embodiments, the kit provides packaged pharmaceutical compositions comprising a pharmaceutically acceptable carrier and compounds of the invention. In certain embodiments the packaged pharmaceutical composition will comprise the reaction precursors necessary to generate the compound of the invention upon combination with a radio labeled precursor. Other packaged pharmaceutical compositions provided by the present invention further comprise indicia comprising at least one of: instructions for preparing compounds according to the invention from supplied precursors, instructions for using the composition to image cells or tissues expressing PSMA, or instructions for using the composition to image glutamatergic neurotransmission in a patient suffering from a stress-related disorder, or instructions for using the composition to image prostate cancer.

D. Pharmaceutical Compositions and Administration

In another aspect, the present disclosure provides a pharmaceutical composition including a presently disclosed theranostic probe or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra -sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### II General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to the presently disclosed compounds are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

More generally, the term “carbocyclic ring” refers to an organic ring structure comprising carbon atoms, which can be aromatic or non-aromatic, and includes cyclopropyl, cyclobutyl, cyclopentyl, cyclohexyl, phenyl, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4- morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight-chain or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-l-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight-chain or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight-chain or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight-chain, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′—and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

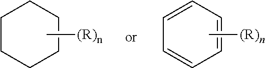

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

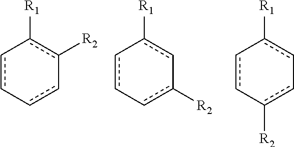

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′,═O,═NR′,═N—OR′, —NR′R″, —SR′, -halogen, —SiR′R″R′″, —OC(O)R′, —C(O)R′, —CO₂R′,′C(O)NR′R″, —OC(O)NR′R″, —NR″C(O)R′, —NR′—C(O)NR″R′″, —NR″C(O)OR′, —NR— C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″ —S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described. The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR' wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

The term “protecting group” in reference to the presently disclosed compounds refers to a chemical substituent which can be selectively removed by readily available reagents which do not attack the regenerated functional group or other functional groups in the molecule. Suitable protecting groups are known in the art and continue to be developed. Suitable protecting groups may be found, for example in Wutz et al. (“Greene's Protective Groups in Organic Synthesis, Fourth Edition,” Wiley-Interscience, 2007). Protecting groups for protection of the carboxyl group, as described by Wutz et al. (pages 533-643), are used in certain embodiments. In some embodiments, the protecting group is removable by treatment with acid. Representative examples of protecting groups include, but are not limited to, benzyl, p-methoxybenzyl (PMB), tertiary butyl (t-Bu), methoxymethyl (MOM), methoxyethoxymethyl (MEM), methylthiomethyl (MTM), tetrahydropyranyl (THP), tetrahydrofuranyl (THF), benzyloxymethyl (BOM), trimethylsilyl (TMS), triethylsilyl (TES), t-butyldimethylsilyl (TBDMS), and triphenylmethyl (trityl, Tr). Persons skilled in the art will recognize appropriate situations in which protecting groups are required and will be able to select an appropriate protecting group for use in a particular circumstance.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments,±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Long-Circulating PSMA-Targeted Phototheranostic Agent

### 1.1 Overview

Targeted photodynamic therapy (PDT) combined with multimodal imaging is an appealing strategy for precision cancer treatment. Targeting the prostate-specific membrane antigen (PSMA) has drawn significant attention due to its marked overexpression in a variety of malignant tissues, most notably prostate cancer. To unleash the potential of targeted PDT, the presently disclosed subject matter provides a long-circulating PSMA-targeted phototheranostic agent (referred to herein as “LC-Pyro”) for multimodal imaging and therapy of prostate cancer.

LC-Pyro comprises three building blocks: (1) a urea-based PSMA-affinity ligand; (2) a peptide linker to prolong plasma circulation time; and (3) a porphyrin photosensitizer for PET/fluorescence imaging and PDT. The multimodal imaging and therapeutic potential of LC-Pyro was validated in several experimental models.

The presently disclosed subject matter demonstrates that LC-Pyro selectively accumulated in PSMA-overexpressing tumors in subcutaneous, orthotopic, and metastatic murine models. The peptide linker in LC-Pyro prolonged its plasma circulation time 8.5-fold compared to an analog containing a single lysine linker, resulting in enhanced tumor accumulation. Inherent metal chelating and optical properties of porphyrins allow for simple transformation of LC-Pyro into a dual modality, fluorescence/PET imaging agent for accurate and quantitative tumor detection. Furthermore, high LC-Pyro tumor accumulation (9.74% ID/g) enabled potent PDT, which resulted in significantly delayed tumor growth with single-dose treatment in a subcutaneous xenograft model.

Accordingly, the presently disclosed strategy significantly extends the plasma circulation of a targeted photosensitizer, which resulted in successful eradication of PSMA-expressing tumors. The presently disclosed approach combined the benefits of a small molecule and a long-circulating antibody-photosensitizer conjugate and can be applied to existing and future targeted PDT agents for improved efficacy.

### 1.2 Background

As provided hereinabove, PDT holds significant promise for treating and managing cancers, including prostate cancer. One way to enhance PDT may be to design a cancer cell-targeted photosensitizer that would generate ROS intracellularly and provide an additional layer of selectivity. Researchers are exploring a variety of small-molecule targeting ligands, antibody-photosensitizer conjugates and targeted nanoparticles for intracellular delivery of photosensitizer. See Abrahamse et al., 2017; Taquet et al., 2007. Such targeting approach has its strengths and limitations.

Small-molecule ligand-photosensitizer conjugates can be designed with high binding affinity to target and are relatively simple to produce, which makes them prime candidates for clinical translation. Chen et al., 2016; Wang et al., 2016. Despite the success of that approach in vitro, rapid renal clearance of the ligand-photosensitizer conjugates may limit their ability to accumulate within tumor, limiting efficacy, regardless of the targeting moiety. See Wang et al., 2017. Nanoparticles can allow for co-delivery of a high payload pf photosensitizer with drugs or imaging agents, however their translation is hindered by high production costs and difficulty in scale-up. See Watanabe et al., 2018; Kumar et al., 2008. Finally, antibody-photosensitizer conjugates offer superb targeting and favorable pharmacokinetics but their utility in solid tumors is often limited by their poor tissue penetration resulting in limited and heterogeneous intratumoral distribution. See Heine et al., 2012.

The presently disclosed subject matter provides a long-circulating photosensitizer (LC-Pyro) that targets the prostate-specific membrane antigen (PSMA) and aims to combine benefits of targeted small molecules and long-circulating photosensitizer-carrying vehicles.

PSMA is a type II transmembrane glycoprotein, which is highly overexpressed in prostate cancer. Its expression correlates with cancer aggressiveness. See Israeli et al., 1994; Bostwick et al., 1998; and Kiess et al., 2016. Recently PSMA has attracted significant attention in the oncology community due to the success of PSMA-targeted nuclear imaging and therapeutic radionuclide delivery, which is beginning to affect management of patients with prostate cancer. Wang et al., 2016; Haberkorn et al., 2016; Kratochwil et al., 2017b; and Kratochwil et al., 2017b.

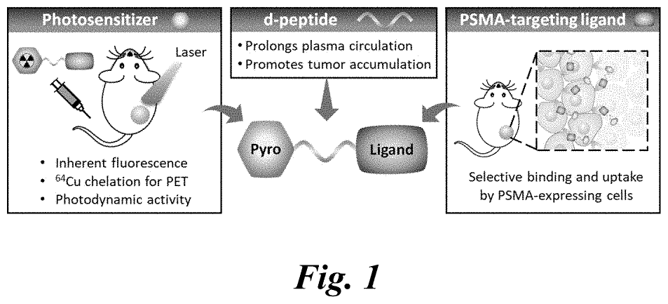

The presently disclosed agent comprises of three building blocks: a highly selective PSMA-binding ligand, a peptide-based pharmacokinetic modulator (see Stefflova et al., 2007) and a pyropheophorbide α photosensitizer (see FIG. 1). Without wishing to be bound to any one particular theory, it is thought that the presence of a peptide linker will prolong the plasma circulation time and enhance tumor accumulation, allowing for an efficient single dose photodynamic treatment, while inherent fluorescence and metal chelating porphyrin properties will allow for multimodal imaging of prostate cancer.

### 1.3 Materials and Methods

The activating agent (benzotriazol-1-yl)-N,N,N′,N′-tetramethyluronium hexafluorophosphate (HBTU) was purchased from Novabiochem (Etobicoke, ON), and used without further purification. The Rink amide resins and all 9H-fluoren-9-ylmethoxycarbonyl (N-Fmoc)-protected amino acids were purchased from Novabiochem. Pyropheophorbide α (Pyro acid) and urea-based PSMA inhibitor containing an N-hydroxysuccinamide (NHS) moiety were synthesized by the previous described protocols. See Zheng et al., 2002; Chandran et al., 2008. Cell culture medium was obtained from ATCC (American Type Culture Collection, Manassas, Va.). FBS and trypsin-ethylenediaminetetraacetic acid (EDTA) solution were purchased from Gibco (Invitrogen Co, Waltham, Mass.). ⁶⁴CuCl₂ was obtained from Washington University (St. Louis, Mo.). Detailed procedures related to synthesis and characterization of LC-Pyro and SC-Pyro, generation of ROS, ligand binding affinity and ⁶⁴Cu radiolabeling are provided herein below.

### 1.3.1 In Vitro Targeting and Cellular Uptake

All cell lines (PSMA+ PC3 PIP, PSMA− PC3 flu, PC3-ML-1124 and PC3-ML-1117) were cultured in RPMI-1640 medium (Invitrogen, Carlsbad, Calif.) containing 10% FBS (Invitrogen) and 1% penicillin-streptomycin (Biofluids, Camarillo, Calif.). See Kiess et al., 2016; Chang et al., 1999. Cell cultures were maintained in a 37° C. humidified incubator under 5% CO₂. PSMA expression for all tested cell lines was validated with western blot analysis. For fluorescence microscopy experiments, PSMA+ PC3 PIP and PSMA− PC3 flu cells were seeded into 8-well coverglass-bottom chambers (Nunc Lab-Tek, Sigma-Aldrich, Rochester, N.Y.) at a cell-seeding density of 2×10⁴ cells per well. After 24 hours of incubation, medium was replaced with 3 μM LC-FITC (1 vol % DMSO in medium) and incubated for 3 hours. For time-dependent imaging studies cells were incubated with LC-FITC for 5 minutes, 10 minutes, 15 minutes, 30 minutes, 1 hours, 3 hours, or 6 hours. For inhibition studies, the known PSMA inhibitor, DCIBzL, was added in molar excess (10, 50, 100×) in combination with 3 μM LC-FITC for 3 hours. Fluorescence imaging was performed on an Olympus IX73 inverted microscope using a 60× magnification objective. LC-FITC signal was detected using a FITC filter (excitation: 485/20 nm bandpass; emission: 522/24 nm) and Hoechst nuclear stain was detected using a DAPI filter (excitation: 387/11 nm bandpass; emission 447/60 nm). Images were processed using ImageJ software. For flow cytometry studies, PSMA+ PC3 PIP and PSMA− PC3 flu cells were seeded in 6-well plates at a cell density of 5×10⁵ cells per well. After 24 hours, cells were replaced with fresh medium and treated with 2 μM LC-FITC for 0.5, 1, 3, 5, and 22 hours. After incubation, cells were trypsinized, centrifuged and washed two times, resuspended in 0.5 mL FACS buffer (0.5 mM EDTA and 5 mg/L DNase in PBS) and filtered. Quantification of the fluorescence signal was performed using a Beckman Coulter FC500 five-color analyzer and FITC fluorescence was detected (FITC channel, excitation: 505 nm LP; emission: 530/30 nm) for 10,000 counts. The exact protocol was applied to LC-Pyro incubation conditions (7AAD channel, excitation: 635 nm LP; emission: 660/20 nm). Median fluorescence was subtracted from cells with no LC-FITC treatment and histogram plots were generated using FlowJo software.

### 1.3.2 Xenograft Mouse Models

Animal studies were conducted under institutional approval (University Health Network, Toronto, Canada). For generation of subcutaneous tumor xenografts, athymic male nude mice under general anesthesia (2 vol % isoflurane in oxygen) were inoculated with 2.5 x 10⁶ PSMA+ PC3 PIP or PSMA− PC3 flu cells in 60 μL of saline into the right or left flank. An orthotopic prostate tumor model was generated as described elsewhere. See Jin et al., 2016. Briefly, 2.5×10⁶ PSMA+ PC3 PIP or PSMA− PC3 flu cells in 20 μL of saline were injected to the dorsal prostate lobe using a 28-gauge needle, animals were sutured back and administered with 0.1 mg/kg of bupenorphine for analgesia. Orthotopic prostate tumor growth was monitored by magnetic resonance imaging (MRI, Biospec 70/30 USR, Bruker, Billerica, Mass.). For the metastatic prostate cancer model, 2×10⁶ PC3-ML-1124 (PSMA+; fluc+) or PC3-ML-1117 (PSMA−; fluc+) cells in 200 μL of saline were administered via lateral tail vein. See Castanares et al., 2016. Development of metastatic nodules was monitored by bioluminescence imaging (Xenogen, Caliper Life Sciences, Hopkinton, Mass.) every three days.

For the targeting studies in vivo, animals bearing PSMA− PC3 flu (left flank) and PSMA+ PC3 PIP (right flank) subcutaneous tumors (n=3) were injected intravenously with 20 nmol of LC-Pyro in 0.2 mL of saline (1 vol % DMSO) and imaged with a CR₁ Maestro imaging system (Caliper Life Sciences, Waltham, Mass.) with 680 nm excitation and 700 nm detection (integration time=500 milliseconds) at 0.5, 1, 3, 6 and 24 hours post-injection. After 24 hours, animals were sacrificed and tumors were excised for ex vivo imaging. For inhibition studies in vivo, animals bearing a single PSMA+ PC3 PIP tumor were injected with 15 nmol of LC-Pyro with a second cohort receiving an intravenous injection of PSMA inhibitor (DCIBzL, 155× molar excess) 30 minutes prior to LC-Pyro injection (n=3). Animals were sacrificed 24 hours post-injection and tumors were excised and imaged ex vivo. Relative difference in fluorescence accumulation was calculated using ImageJ software.

### 1.3.3 Pharmacokinetics and Qualitative Fluorescence Biodistribution Studies

For the pharmacokinetic studies, LC-PSMA, SC-PSMA and YC-9, see Chang et al., 1999, (n=5 each cohort), were intravenously injected into healthy BALB/c mice at the dose of 20 nmol per animal. Blood was collected from the saphenous vein prior to injection of probe and also at 5 minutes, 0.5 hours, 1 hours, 2 hours, 4 hours, 8 hours, 24 hours, and 48 hours post-injection. Blood samples were then centrifuged for 10 minutes at 10,000 rpm and the collected plasma fraction was diluted 50× in DMSO. Fluorescence emission for Pyro (616- to 661-nm bandpass excitation and 675-nm longpass emission optical interference filters integration time=500 milliseconds) and YC-9 (excitation: 620 nm; emission: 650-850 nm) was measured by Fluoromax-4 spectrofluorometer (Horiba Scientific, N.J.) and normalized integrated peak values were analyzed by Graphpad Prism to calculate the half-life of each compound. For qualitative biodistribution studies animals bearing dual PSMA PC3 PIP and PSMA− PC3 flu subcutaneous tumors were injected with LC-Pyro or SC-Pyro (20 nmol) and images were analyzed using software from the CR₁ Maestro imaging system.

### 1.3.4 PET/CT Imaging and Biodistribution of Mouse Xenografts

Mice bearing PSMA PC3 PIP (n=4) or PSMA− PC3 flu (n=3) orthotopic prostate tumors were administered with ⁶⁴Cu-LC-Pyro solution (0.2 m1, 1 vol % DMSO, approximately 0.5 mCi, 25 nmol of LC-Pyro). PET/CT imaging was performed on one animal from each group at 3 hours and 17 hours post-injection on a small-animal MicroPET system (Focus 220: Siemens, Munich, Germany) with CT co-registration on a microCT system (Locus Ultra: GE Healthcare, U.K.). Twenty-four hours post-injection of ⁶⁴Cu-LC-Pyro, animals were euthanized via cardiac puncture under 2% isoflurane anesthesia. Organs of interest, including the tumor, prostate, seminal vesicles, testes, heart, spleen, lungs, liver, kidneys, adrenal, stomach, small intestine, large intestine, skin, fat, muscle, bone and brain were excised, washed in saline, dried with absorbent tissue, weighed, and counted on a gamma-counter Wizard® 1480 well-type automatic gamma counter (PerkinElmer Inc.; Shelton, Conn.). Radioactivity measured in each organ was decay-corrected and expressed as the percentage of the injected dose per gram of tissue (% ID/g).

### 1.3.5 Fluorescence Detection of Prostate Metastasis

Animals with PSMA+ (PC3-ML-1124) or PSMA− (PC3-ML-1117) firefly luciferase-expressing metastatic lesions were administered with 20 nmol of LC-Pyro (0.2 mL saline, 1 vol % DMSO) via tail vein injection. Twenty-four hours post-injection animals received 60 mg/kg of luciferin intraperitoneally and sacrificed 10 minutes later via cervical dislocation. Internal organs were removed to expose the metastatic nodules present in retroperitoneal cavity alongside bioluminescent imaging to detect the exact location of the metastases. Fluorescent imaging was conducted in situ and ex vivo using a CRi Maestro imaging system (616-nm to 661-nm bandpass excitation and 675-nm longpass emission optical interference filters; integration time=500 milliseconds). The nodules were excised with the surrounding muscle tissue, placed in optical cutting temperature (OCT) compound and snap-frozen in liquid nitrogen vapor. Sections at 10-μm thickness were cut, deparaphinized and stained with DAPI-containing mounting medium. Fluorescence imaging of tissue slices was performed on an Olympus IX73 inverted microscope using a 20× magnification objective. LC-Pyro signal was detected using a Cy5 filter (excitation: 628 nm/40 nm bandpass; emission: 692 nm/40 nm) and DAPI signal was detected using a DAPI filter (excitation: 387 nm/11-nm bandpass; emission: 447 nm/60 nm). Images were pseudocolored and the intensity was scaled using CellSens software (Olympus Canada Inc.).

### 1.3.6 Photodynamic Therapy (PDT)

Mice bearing one subcutaneous PSMA+ PC3 PIP flank tumor were randomly divided into four groups: (1) saline only, (2) LC-Pyro only, (3) laser only and (4) LC-Pyro+laser (n=4). Animals in group 2 and group 4 were intravenously injected with 50 nmol and 30 nmol of LC-Pyro, respectively. LC-Pyro uptake was monitored by in vivo fluorescence imaging for 6 hours, followed by tumor laser treatment for mice in groups 3 and 4. Using a 671-nm free-space laser (DPSS LaserGlow Technologies), tumors were treated with a single PDT light dose of 100 J/cm² (0.63 cm² laser spot area; fluence rate 55 mW/cm²). Body weight was recorded and tumor volumes were measured with calipers using the equation Vₜᵤₘₒᵣ=½ (length×width×width). Animals were removed from the experiment and sacrificed when the tumor reached>1,000 mm³ or started ulcerating.

### 1.3.7 Statistical Analysis

A two-tailed Student's t test was used to determine statistical significance. P-values less than 0.05 were considered significant.

### 1.3.8 Synthesis and Characterization of Pyro-Peptide-PSMA (LC-Pyro) and Pyro-k-PSMA (SC-Pyro)

A peptide sequence with D amino acid backbone, Fmoc-gd(OtBu)e(OtBu)vd(OtBu)gs(tBu)gk(Mtt), was synthesized on Rink resin using Fmoc chemistry protocol. After removing the last Fmoc group, Pyro acid was coupled to the N-terminal of the peptide on resin at room temperature ([Pyro acid/HBTU/peptide 3:3:1]). The

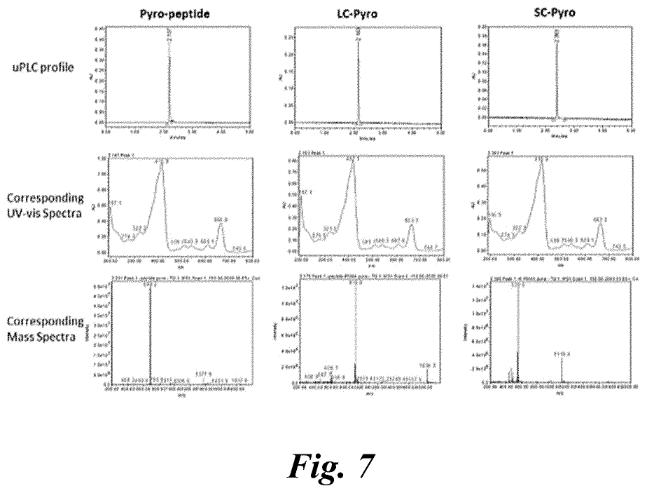

Pyro-peptide-resin was then treated with a cleavage cocktail (TFA: triisopropylsilane: water=95:2.5:2.5) for 1 hour at room temperature to remove the resin and cleave the protected groups. The acquired Pyro-peptide (Pyro-GDEVDGSGK(NH₂)) was conjugated with PSMA-NHS (Pyro-peptide/PSMA-NHS/DIPEA, 1:1.2:2) in anhydrous DMSO. The acquired Pyro-peptide-PSMA (LC-Pyro) was purified by HPLC. Pyro-k-PSMA (SC-Pyro) was synthesized in a similar way with the peptide linker replaced by a single lysine linker. The synthesis of LC-Pyro and SC-Pyro were confirmed with uPLC-MS analysis with identified ESI mass spectrometry and corresponding UV-vis absorption (FIG. 7). LC-Pyro (m/z calculated for C₈₆H₁₁₈N₁₈O₂₇ [M]⁺ 1835.99, found [M]⁺ 1836.3, [M]²⁺ 918.0); SC-Pyro (m/z calculated for C₅₉H₇₈N₁₀O₁₂) [M]⁺1119.33, found [M]⁺ 1119.4, [M]²⁺ 559.6). Reverse-phase analytical uPLC-MS were performed on a ACQUITY UPLC® BEH C18 column (1.7 μm, 2.1 mm×50 mm) using a Waters 2695 controller with a 2996 photodiode array detector and a Waters TQ mass detector. The conditions were as follows: solvent A) 0.1% trifluoroacetic acid (TFA); B) acetonitrile; column temperature: 60° C.; flow rate: 0.6 mL/min gradient: from 60% A+40% B to 0% A+100% B in 3 minutes, kept at 100% B for 1 minute, followed by a sharp change back to 60% A+40% B and a hold for another 1 minute. A fluorescence spectrum of LC-Pyro was acquired on a Fluoromax-4 fluorometer (Horiba Jobin Yvon, N.J.).

### 1.3.9 Reactive Oxygen Species Generation of LC-Pyro

Reactive oxygen species generation of LC-Pyro was measured using a commercially available Amplex UltraRed Reagent (AUR) assay (Thermo Fisher Scientific). The OD₆₆₅ₙₘ of LC-Pyro in 70:30 MeOH:PBS was set to 0.15 and was added in a black clear-bottom 96-well plate. AUR was dissolved in DMSO (10 mM) and diluted 100-fold in each well. The wells were then irradiated by a 671-nm laser (DPSS LaserGlow Technologies) at increasing light doses (0.5, 1.0, 2.0, 3.0 and 5.0 J/cm²). Fluorescence emission of the fluorogenic product of AUR was measured (excitation: 550 nm; emission: 581 nm) using a ClarioStar microplate reader (BMG LABTECH).

### 1.3.10 Ligand Binding Affinity of LC-Pyro, SC-Pyro and DCIBzL

The inhibitory activities of LC-Pyro, SC-Pyro and DCIBzL against PSMA were determined using a fluorescence-based assay according to a previously reported procedure. Chen et al., 2009. Briefly, lysates of LNCaP cells (25 μL in 0.1 M Tris-HCl, pH 8.0) were incubated with the serial dilutions of the test compounds (in 12.5 μL of 0.1 M Tris-HCl, pH 8.0) in the presence of 4 μM N-acetylaspartylglutamate (NAAG) (in 12.5 μL of 0.1 M Tris-HCl, pH 8.0) for 120 minutes. The reaction mixtures were incubated with the working solution (50 μL) of the Amplex Red Glutamic Acid Kit (Molecular Probes Inc., Eugene, Oreg.) for 60 minutes. The amount of glutamate released from NAAG hydrolysis by the LNCaP lysates was determined by measuring the fluorescence generated from the reactions using the Cytation 5 plate reader (BioTek, Winooski, Vt.) with excitation at 545 nm and emission at 590 nm. Inhibition curves were determined using semi-log plots, and IC₅₀ values were determined at the concentration at which enzyme activity was inhibited by 50%. Enzyme inhibitory constants (Kᵢ values) were generated using the Cheng-Prusoff conversion. Cheng et al., 1973. Data analysis was performed using GraphPad Prism software (n=3).

### 1.3.11 ⁶⁴Cu Radiolabeling

LC-Pyro (225 nmol) was dissolved in 18 μL of DMSO and saline was added then vortexed, producing a dark green solution (450 μM). ⁶⁴Cu(Cl)₂ solution (pH=5.5; approximately 5.0 mCi; 0.5 mL) was then added and the reaction mixture was heated in a water bath at 60° C. for 30 minutes. After radiolabeling was completed, sample was diluted with 1 mL of saline and the radiochemical purity was analyzed by instant thin layer chromatography (iTLC). Briefly, a 1-cm by 8-cm strip of heat-activated glass microfiber chromatography paper (Aligent Technologies, USA) was spotted with 24 of sample 1.5-cm from the bottom of the strip. The strip was then placed into a capped test tube containing mobile phase prepared with 2 vol % EDTA (Sigma-Aldrich Co. LLC) and 10 vol % 0.1 M NH₄OAc in ddH₂O. The retention value of non-chelated ⁶⁴Cu was reproducibly greater than 0.9. The developed iTLC strip was cut in thirds and the ⁶⁴Cu radioactivity assayed for the two top (free ⁶⁴Cu) and separately for a bottom piece (⁶⁴Cu-LC-Pyro) using a Wizard® 1480 well-type automatic gamma counter (PerkinElmer Inc.; Shelton, Conn., USA) and radiochemical purity was further evaluated by radio HPLC performed on a XBridge-C18 column (2.5 μm, 4.6 mm×50 mm) with UV detector and radioactivity detector (FIG. 7).

Further purification of ⁶⁴Cu-LC-Pyro was deemed unnecessary, due to the high molar ratio of LC-Pyro:⁶⁴CuC12 (approximately 1000:1) and its high radiochemical purity. In addition, previous reports indicate that copper chelation into a porphyrin ring did not significantly change the in vivo uptake and clearance profiles. Wilson et al., 1988.

### 1.4 Results

### 1.4.1. LC-Pyro Synthesis and Characterization

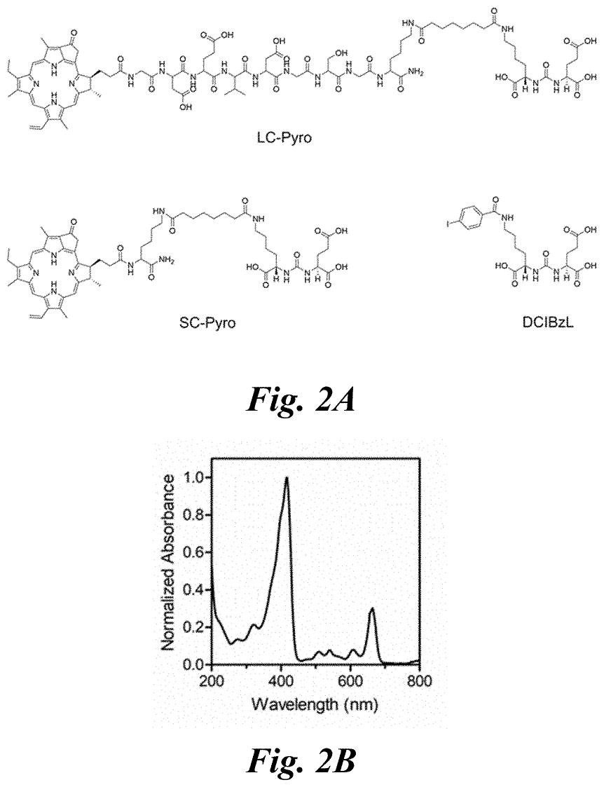

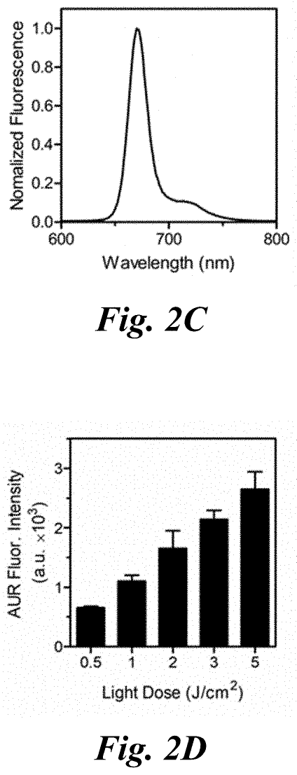

The presently disclosed PSMA-targeted phototheranostic agent that consists of three functional building blocks: (1) a porphyrin-based photosensitizer capable of multimodal fluorescence/PET imaging and photodynamic activity; (2) a 9-amino acid D-peptide linker to impart water-solubility and improve the plasma circulation time; and (3) a urea-based high-affinity PSMA targeting ligand (FIG. 1). LC-Pyro (Long-circulation pyropheophorbide α) and SC-Pyro (Short-circulating pyropheophorbide α) were synthesized and confirmed by uPLC-MS analysis with identified ESI+ mass spectrometry and corresponding UV-vis absorption. (FIG. 2A and FIG. 7). LC-Pyro (m/z calcd [M]⁺ 1835.99, found [M]⁺1836.3, [M]²⁺ 918.0); SC-Pyro (m/z calcd [M]⁺ 1119.33, found [M]⁺ 1119.4, [M]²⁺ 559.6). DCIBzL was previously synthesized and was used as an inhibitor ligand in vitro and in vivo to confirm PSMA specificity. See Chandran et al., 2008. LC-Pyro absorbance (FIG. 2B) and fluorescence (FIG. 2C) were collected and its measured photodynamic activity revealed an increase in generation of ROS with an increase in laser light dose up to 5 J/cm² (FIG. 2D).

### 1.4.2 LC-Pyro Demonstrates High Selectivity and Specificity In Vitro

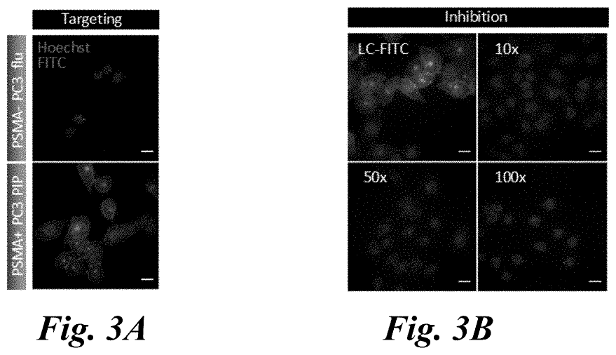

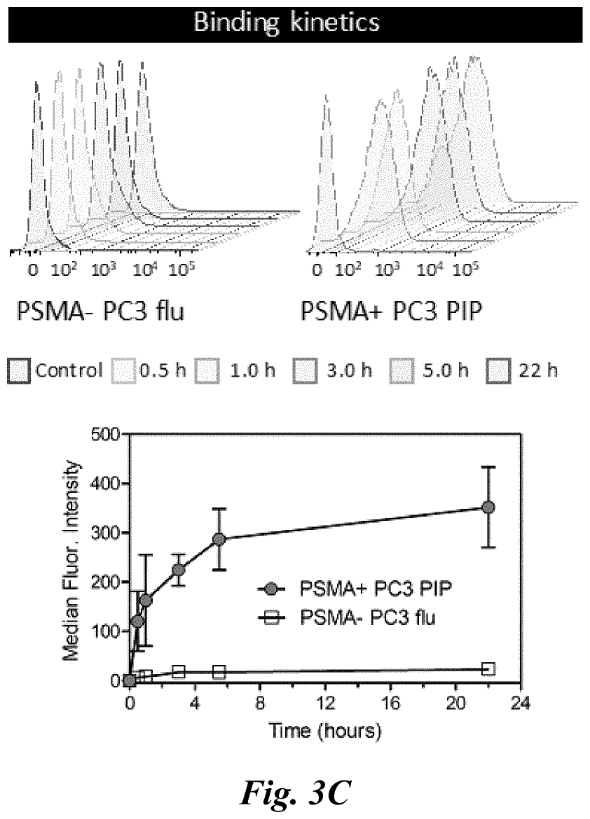

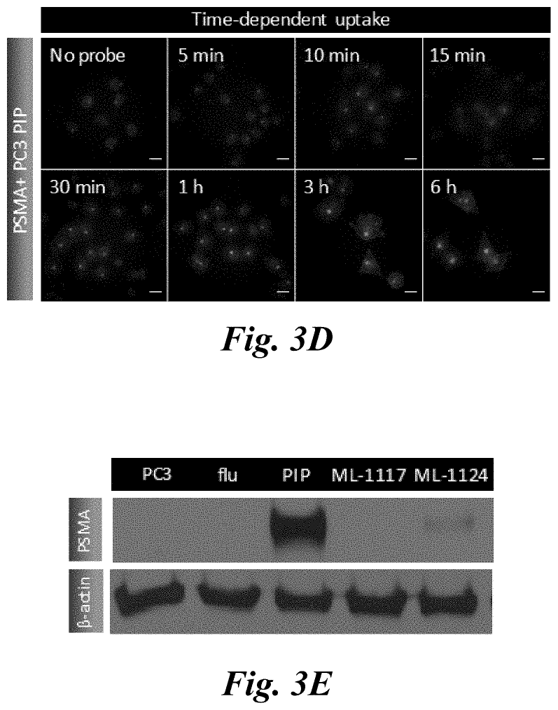

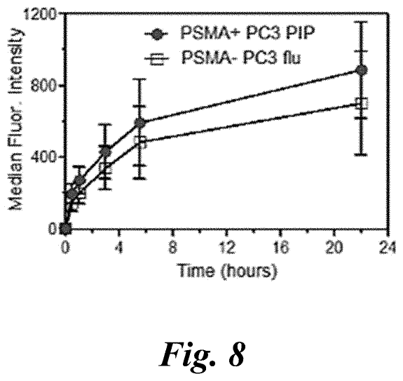

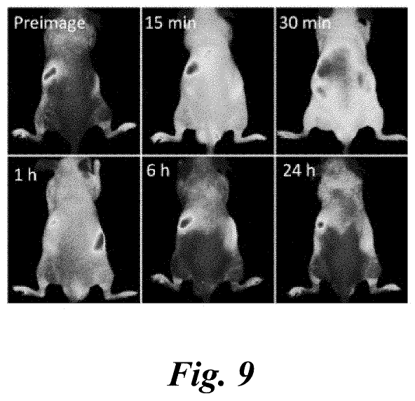

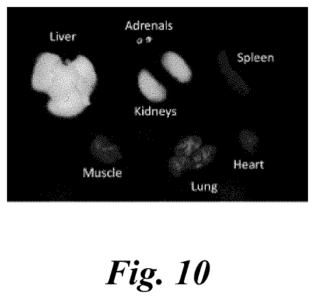

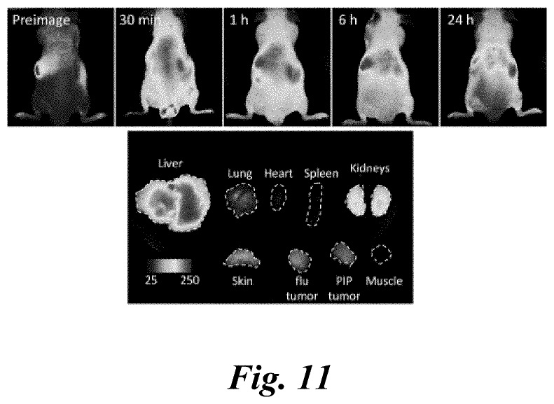

Targeted uptake of a photosensitizer by biomarker-overexpressing cancer cells can introduce an additional level of selectivity for PDT. Due to the inherent cell-penetrating properties of porphyrins in vitro, see Chen et al., 2005, a FITC-labeled analog (LC-FITC) was used to investigate the targeting selectivity of the peptide-PSMA moiety. Fluorescence microscopy in FIG. 3A revealed selective membrane staining in PC3 prostate cells overexpressing PSMA (PSMA+ PC3 PIP), whereas negligible fluorescence was observed in cells with low PSMA expression (PSMA− PC3 flu) under equivalent incubation settings (3 μM, 3 hours) and exposure time. LC-FITC also localized to one focus within the perinuclear region, which has been observed previously and described to represent the mitotic spindle poles or an endosomal compartment. See Kiess et al., 2015. Addition of excess DCIBzL to LC-FITC achieved successful PSMA binding inhibition as low as 10-fold, indicating target specificity of the conjugated small-molecule ligand (FIG. 3B). LC-FITC selectivity was confirmed by flow cytometry over a 22-hour period. Fluorescence intensity from LC-FITC uptake increased in a time-dependent manner in PSMA+ PC3 PIP cells with a 15-fold higher uptake than PSMA− PC3 flu cells (FIG. 3C). Flow cytometry conducted with LC-Pyro confirmed the nonselective cell-penetrating properties of the porphyrin (FIG. 8). Cell uptake of LC-FITC in PSMA+ PC3 PIP cells was also assessed using fluorescence microscopy, which corresponded well with the cytometric measurements (FIG. 2D). Western blot in FIG. 3E validated PSMA expression in the primary or metastatic (ML) lines modified to express high (PSMA+ PC3 PIP; PC3-ML-1124) or low (PSMA− PC3 flu; PC3-ML-1117) levels of PSMA.

### 1.4.3 Peptide Linker Prolongs Plasma Circulation Time Enhancing Tumor Accumulation of LC-Pyro

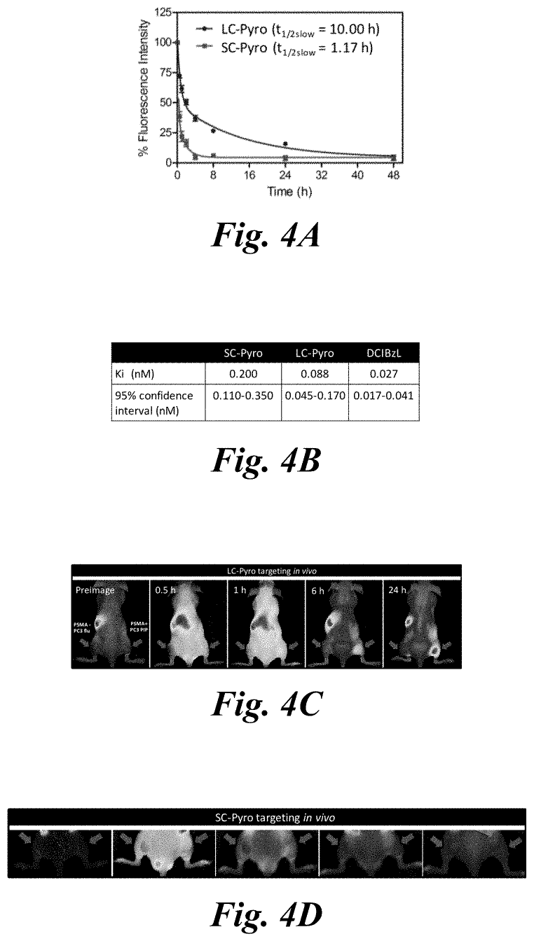

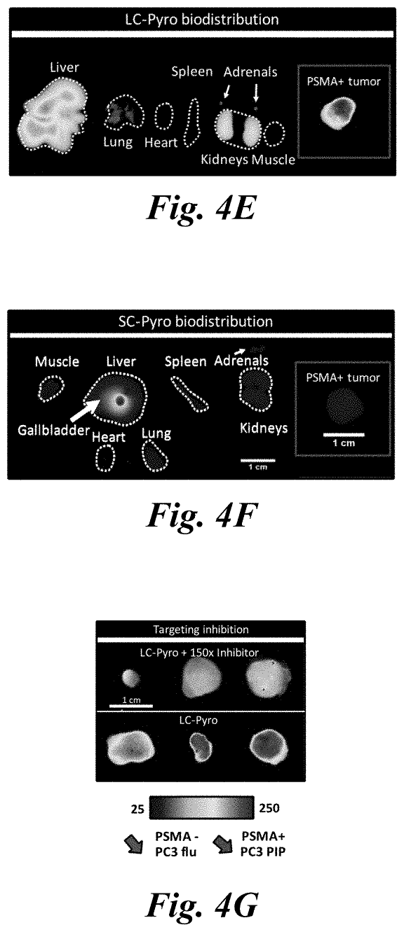

To validate the pharmacokinetic role of the peptide linker in LC-Pyro and its ability to accumulate in PSMA+ tumors, a truncated derivative, SC-Pyro, was synthesized where the 9-amino acid peptide sequence was replaced by a single lysine residue. Results in FIG. 4A demonstrated that LC-Pyro exhibited an 8.5-fold longer plasma circulation time (t_{1/2, slow=}10.00 hours; t_{1/2, fast}=0.50 hours) than SC-Pyro (t_{1/2, slow}=1.17 hours; t_{1/2, slow}=0.20 hours). To ensure that the in vivo targeting results were not affected by any difference in binding affinity, the inhibitory activities of LC-Pyro, SC-Pyro and DCIBzL against PSMA were determined using a fluorescence-based assay. All three compounds demonstrated sub-nanomolar inhibitory capacity. Conjugation of a porphyrin peptide linker to the PSMA inhibitor did not significantly alter its PSMA inhibitory properties (FIG. 4B). Next, to assess the influence of the plasma circulation time between LC-Pyro and SC-Pyro on tumor accumulation and biodistribution, equivalent doses of each agent were intravenously administered to mice bearing dual subcutaneous PSMA− PC3 flu and PSMA+ PC3 PIP tumors followed by whole-animal in vivo fluorescence imaging over 24 hours. FIG. 4C shows a strong diffuse fluorescence signal from LC-Pyro after 1 hour with selective accumulation to the PSMA+ after 24 hours. There is negligible accumulation within both the PSMA− and PSMA+ tumors 24 hours post-administration of SC-Pyro (FIG. 4D), indicating the importance of long plasma circulation time for successful tumor accumulation. Ex vivo organ distribution in FIG. 4E reveals liver and kidney clearance of LC-Pyro, whereas a majority of SC-Pyro was metabolized and collected in the gallbladder (FIG. 4F). That observation suggests that SC-Pyro cleared rapidly from the body, and therefore, a much higher dose or multiple doses would be required to achieve comparable tumor accumulation to LC-Pyro after 24 hours. Finally, the specificity of LC-Pyro to PSMA was further confirmed in the xenograft model by inhibition with excess DCIBzL. Excised PSMA+ PC3 PIP tumors revealed less accumulation of LC-Pyro when DCIBzL (PSMA blocker) was injected 30 minutes beforehand (FIG. 4G).

### 1.4.4 Multimodal Imaging of PSMA+ Prostate Cancer

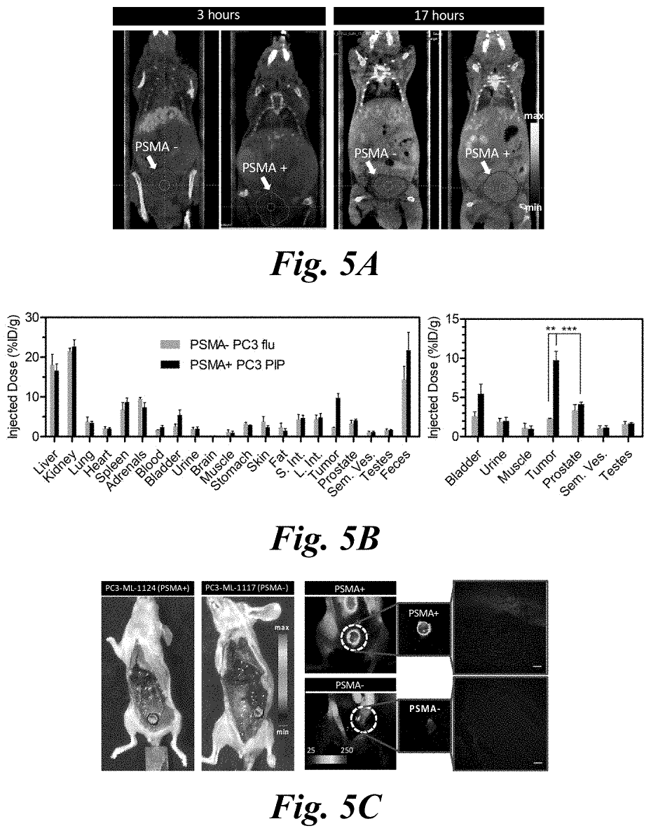

After validating targeting of LC-Pyro in a subcutaneous PSMA+ PC3 PIP tumor model, we explored its theranostic application in two different mouse models bearing either a primary prostate tumor or metastatic prostate cancer. Due to the intrinsic metal-chelation properties of porphyrin ring structures, LC-Pyro was chelated to ⁶⁴Cu, which allowed for in vivo PET imaging and biodistribution. See Shi et al., 2011. As demonstrated in FIG. 5A with PET/CT imaging, ⁶⁴Cu-LC-Pyro delineated the orthotopic PSMA+ PC3 PIP tumor 17 hours post-injection, whereas the PSMA− PC3 flu tumor did not have significant uptake. Biodistribution revealed over 4-fold selective accumulation in PSMA+ PC3 PIP tumor [9.74±2.26 percentage injected dose per gram of tissue (% ID/g)] vs. 2.30±0.09% ID/g in control (FIG. 5B). ⁶⁴Cu-LC-Pyro accumulated greatest in the liver, kidney and feces, indicative of hepatobiliary and renal clearance. The corresponding ⁶⁴Cu-LC-Pyro biodistribution from FIG. 5 of main organs and PSMA− PC3 flu and PSMA+ PC3 PIP tumors quantified via gamma counting is presented in Table 2.

To investigate further the potential of LC-Pyro to accumulate selectively in PSMA-expressing malignant tissues, fluorescence imaging of micrometastasis was performed. In situ fluorescence images of fluc+/PSMA+ (PC3-ML-1124) and fluc+/PSMA− (PC3-ML-1117) tumors demonstrated selective uptake of LC-Pyro in the PSMA+ tumor nodule (FIG. 5C). Those observations aligned with the previous data obtained in subcutaneous xenografts. Following in situ fluorescence imaging, further verification of LC-Pyro accumulation in metastatic nodules and the surrounding tissues was examined by fluorescence microscopy of DAPI-stained tissue sections. Robust porphyrin fluorescence signal in the PSMA+ metastatic nodule microstructure was observed. Notably, the surrounding muscle tissue demonstrated fluorescence near to that of background, further supporting PSMA+ cell selectivity of LC-Pyro.

TABLE 2
⁶⁴Cu-LC-Pyro Biodistribution Quantified by Gamma Counting

| Organ | PSMA − PC3 flu | PSMA + PC3 PIP |
|---|---|---|
| Liver | 18.11 ± 4.43 | 16.61 ± 3.39 |
| Kidney | 21.52 ± 1.27 | 22.68 ± 3.38 |
| Lung | 3.81 ± 1.98 | 3.47 ± 0.76 |
| Heart | 2.00 ± 0.83 | 1.99 ± 0.45 |
| Spleen | 6.85 ± 2.93 | 8.68 ± 2.10 |
| Adrenals | 9.55 ± 0.65 | 7.36 ± 2.38 |
| Blood | 1.74 ± 0.03 | 2.35 ± 0.92 |
| Bladder | 1.92 ± 0.71 | 5.45 ± 2.51 |
| Urine | 2.60 ± 1.00 | 2.00 ± 0.92 |
| Brain | 0.16 ± 0.02 | 0.18 ± 0.03 |
| Muscle | 1.13 ± 0.98 | 0.98 ± 0.77 |
| Stomach | 3.25 ± 0.63 | 2.85 ± 0.22 |
| Skin | 3.81 ± 2.18 | 2.38 ± 0.69 |
| Fat | 2.32 ± 1.88 | 1.49 ± 1.12 |
| S. Intestine | 4.33 ± 2.08 | 4.70 ± 1.39 |
| L. Intestine | 4.44 ± 1.69 | 4.84 ± 1.39 |
| Tumor | 2.30 ± 0.09 | 9.74 ± 2.26 |
| Prostate | 3.38 ± 1.22 | 4.14 ± 0.53 |
| Sem. Ves. | 1.09 ± 0.53 | 1.16 ± 0.47 |
| Testes | 1.59 ± 0.57 | 1.66 ± 0.26 |
| Feces | 14.35 ± 5.79 | 21.74 ± 8.96 |

(n = 4 for PSMA + PC3 PIP; n = 3 for PSMA − PC3 flu and PSMA, **P < 0.01; **P < 0.001).
Data are represented as mean ± SD.

1.4.5 PDT with LC-Pyro Results in Significantly Delayed Tumor Growth

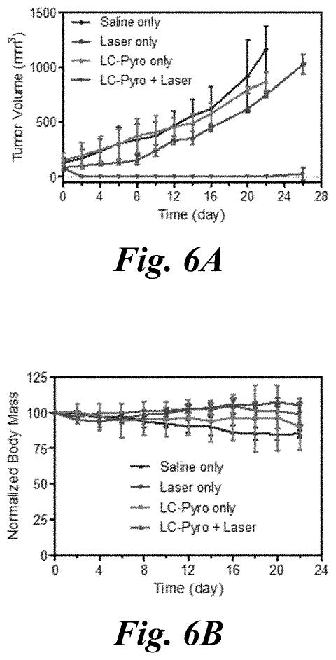

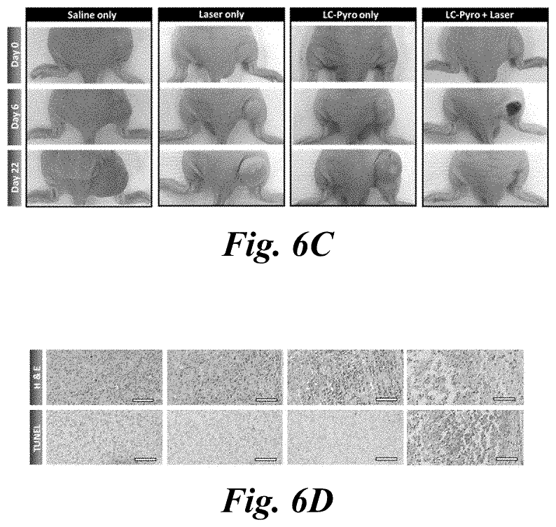

The therapeutic potential of LC-Pyro was evaluated by performing in vivo LC-Pyro-enabled PDT in the PSMA+ PC3 PIP subcutaneous tumor model. Optimization (data not shown) indicated a dose of 100 J/cm² fluence to be appropriate, which is within the clinically relevant dose range. In mice injected with LC-Pyro and treated by light, significant swelling was observed in the tumor region 24 hours after PDT. Approximately four days after treatment, mice in the LC-Pyro+Laser group developed scarring in the tumor region, which was completely healed by day 22 (FIG. 6C). No therapeutic effect was observed in the animals treated with saline, LC-Pyro without laser exposure or laser alone. Importantly, animals injected with LC-Pyro with no laser treatment revealed no sign of skin phototoxicity upon daylight exposure. Overall, significant tumor growth inhibition was observed in the LC-Pyro+Laser group compared to the control cohorts (FIG. 6A), with no decrease in body weight (FIG. 6B). After day 22 tumor regrowth was observed in two animals, most likely from an insufficient laser irradiation area, limited by the maximum beam spot diameter in the custom-built optical setup. The two remaining animals demonstrated no signs of residual or recurrent disease 44 days post-PDT.

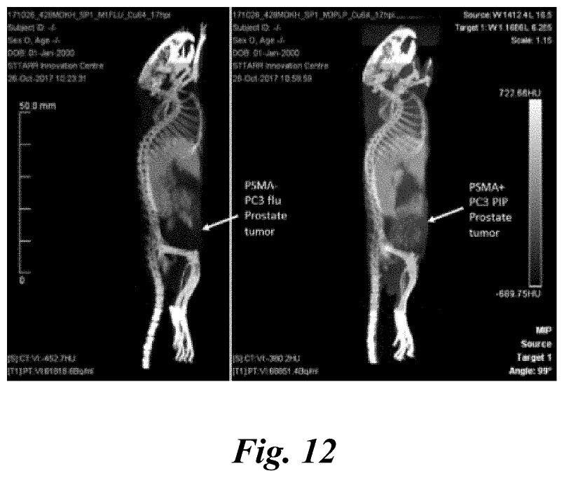

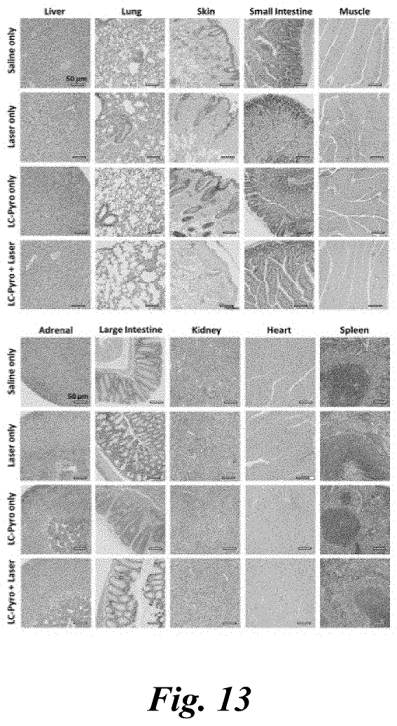

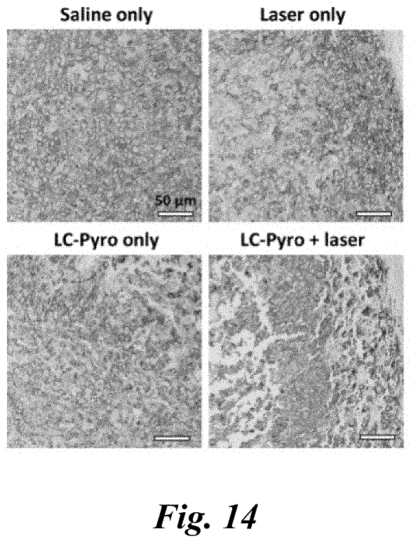

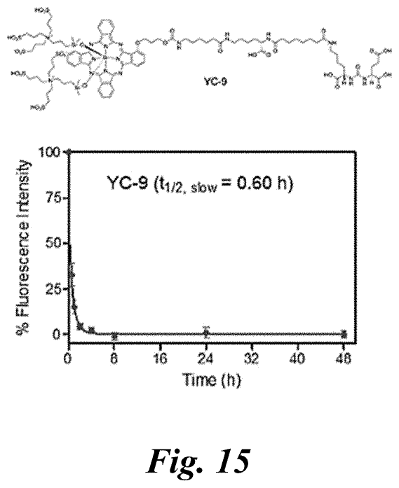

Acute cytotoxic effects of PSMA-targeted PDT were confirmed in a separate animal cohort, where PSMA+ PC3 PIP subcutaneous tumors and organs were harvested 24 hours post-PDT treatment. H&E staining revealed significant damage of tissue architecture in the LC-Pyro+Laser group, while tumors harvested from the other control groups revealed high tumor cell density and intact cellular structure (FIG. 5D). TUNEL staining confirmed the presence of significant cell death in the LC-Pyro+Laser group. No acute damage to major organs was observed as confirmed by histology (FIG. 13). PDT did not significantly alter expression of PSMA (FIG. 14).

### 1.5 Discussion

Significant challenges remain for patients diagnosed with prostate cancer, including those with localized disease. See Serrell et al., 2017. Incomplete tumor eradication of localized disease confers a risk of developing metastases. See Fakhrejahani et al., 2017. Given the multifaceted nature of prostate cancer, precision strategies are necessary. Targeted PDT in combination with pre- and intraoperative imaging promises selective tumor eradication with minimal adverse effect on adjacent off-target tissues.

Using a variety of strategies, several PSMA-targeted photosensitizers have appeared recently. See Chen et al., 2016; Wang et al., 2016; Serrell et al., 2017; Fakhrejahani et al., 2017; Liu et al., 2009; Liu et al., 2010; Nagaya et al., 2017. For example, Chen et al. developed a small-molecule PSMA-targeted photosensitizer, see Chen et al., 2017, while Nagaya et al. conjugated a near-infrared photosensitizer to a monoclonal antibody. See Nagaya et al., 2017. While the low-molecular-weight photosensitizer enables deep tumor tissue penetration and fast targeting kinetics, its rapid clearance resulted in a suboptimal efficacy, explaining the need for four cycles of PDT. On the contrary, the antibody-based photosensitizer exhibited a long circulation time and favorable biodistribution, See Nagaya et al., 2017, however, poor tissue penetration limited its therapeutic potential. See Minchinton and Tannock, 2006. To address limitations of existing PSMA-targeted photosensitizers, an agent was designed that combines the virtues of low molecular weight (<2 kDa) and synthetic accessibility demonstrated by small molecules, while maintaining the long circulation time characteristic of antibody-photosensitizer conjugates. The presently disclosed subject matter demonstrates that the insertion of a peptide between a porphyrin photosensitizer and a PSMA-targeting small-molecule ligand (LC-Pyro) extends its plasma circulation time 8.5-fold in comparison to an analogous derivative containing a single lysine linker (SC-Pyro). That allowed for repeated passages of the agent through the tumor vasculature, increasing the probability of extravasation and further PSMA binding and cell internalization. As a result, this approach achieved suitable LC-Pyro tumor uptake (9.74% ID/g) after a single intravenous administration, eliminating the need for repeated injections. Furthermore, comparing to larger antibody-photosensitizer conjugates, the relatively low molecular weight of LC-Pyro should enable diffusion through the tumor interstitium reaching deep within the tumor.

PSMA targeting is becoming increasingly practiced for prostate cancer detection, image-guided surgical resection and targeted delivery of radiopharmaceuticals. See Liu et al., 2017; Neuman et al., 2015. For example, the PSMA-targeted PET agent, ¹⁸F-DCFBC, has been evaluated in a phase I/II clinical trial for primary prostate cancer and showed higher specificity in detecting clinically significant, high-grade tumors compared to the standard of care, multiparametric MR imaging. See Rowe et al., 2015. Other such trials are also proliferating worldwide. Szabo et al., 2015; Giesel et al., 2018; and Hofman et al., 2018. Furthermore, PSMA-targeted delivery of beta-, and more recently alpha-particle emitters has demonstrated image-based tumor regression in a number of cases. See Kratochwil et al., 2017a; Kratochwil et al., 2017b. Due to the intrinsic metal-chelating feature of porphyrin, LC-Pyro can be readily radiolabeled with positron-emitters, such as ⁶⁴Cu, allowing for non-invasive and quantitative assessment of PSMA expression and treatment planning. Deep-red fluorescence of pyropheophorbide a could also provide guidance for therapeutic interventions. A similar strategy to that demonstrated here could be universally applied to the design of photosensitizers targeting alternative cancer-specific biomarkers beyond PSMA. Finally, the use of a pharmacokinetic modulator to extend the plasma circulation time of the photosensitizer may enhance the efficacy of other targeted cancer treatment strategies, such as radioimmunotherapy and the use of agents activatable within the tumor microenvironment.

### 1.6 Summary

LC-Pyro is a versatile, long-circulating, PSMA-targeted phototheranostic agent. The embedded peptide linker extended its plasma circulation time up to 10.00 hours compared to its truncated derivative (1.17 hours), resulting in increased tumor accumulation (9.74% ID/g). Favorable pharmacokinetics and of LC-Pyro in combination with its targeted PSMA binding led to the effective single-dose tumor ablation by PDT in a PSMA+ PC3 PIP subcutaneous mouse model. Radiolabeling of LC-Pyro with ⁶⁴Cu enabled PET imaging, which can be used for precision treatment planning. LC-Pyro also proved effective for fluorescence-based detection of PSMA+ metastatic nodules, which is important for image-guided surgical resection or palliative PDT.

### REFERENCES

All publications, patent applications, patents, and other references mentioned in the specification are indicative of the level of those skilled in the art to which the presently disclosed subject matter pertains. All publications, patent applications, patents, and other references (e.g., websites, databases, etc.) mentioned in the specification are herein incorporated by reference in their entirety to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Cengel, Keith A S C B, Glatstein E. P D T: What's Past Is Prologue. Cancer Res. 2016;76:2497-500.

Wilson B C, Patterson M S. The physics, biophysics and technology of photodynamic therapy. Phys Med Biol. 2008;53:R₆₁-109.

Plaetzer K, Krammer B, Berlanda J, Ben F. Photophysics and photochemistry of photodynamic therapy: fundamental aspects. Lasers Med Sci. 2009;24:259-68.

Celli J P, Spring B Q, Rizvi I, Evans C L, Samkoe K S, Verma S, et al. Imaging and photodynamic therapy: Mechanisms, monitoring, and optimization. Chem Rev. 2010;110:2795-838.

Mallidi S, Spring B, Hasan T. Optical Imaging, Photodynamic Therapy and Optically-Triggered Combination Treatments. 2016;8:583-92.

Abrahamse H, Hamblin M R. New photosensitizers for photodynamic therapy. 2016;347-64.

Trachtenberg J, Bogaards A, Weersink R A, Haider M A, Evans A, McCluskey S A, et al. Vascular Targeted Photodynamic Therapy With Palladium-Bacteriopheophorbide Photosensitizer for Recurrent Prostate Cancer Following Definitive Radiation Therapy: Assessment of Safety and Treatment Response. J Urol. 2007;178:1974-9.

Taneja S S, Bennett J, Coleman J, Grubb R, Andriole G, Reiter R E, et al. Final Results of a Phase I/II Multicenter Trial of WST11 Vascular Targeted Photodynamic Therapy for Hemi-Ablation of the Prostate in Men with Unilateral Low Risk Prostate Cancer Performed in the United States. J Urol. 2016;196:1096-104.

Trachtenberg J, Weersink R A, Davidson S R H, Haider M A, Bogaards A, Gertner M R, et al. Vascular-targeted photodynamic therapy (padoporfin, WST09) for recurrent prostate cancer after failure of external beam radiotherapy: A study of escalating light doses. B J U Int. 2008;102:556-62.

Azzouzi A R, Vincendeau S, Barret E, Cicco A, Kleinclauss F, van der Poel H G, et al. Padeliporfin vascular-targeted photodynamic therapy versus active surveillance in men with low-risk prostate cancer (CLIN1001 PCM301): an open-label, phase 3, randomised controlled trial. Lancet Oncol. 2017;18:181-91.

Abrahamse H, Hamblin M R, Africa S, Hospital M G. New photosensitizers for photodynamic therapy. Biochem J. 2017;473:347-64.

Taquet J, Frochot C, Manneville V, Barberi-heyob M. Phthalocyanines Covalently Bound to Biomolecules for a Targeted Photodynamic Therapy. 2007;1673-87.

Chen Y, Chatterjee S, Lisok A, Minn I, Pullambhatla M, Wharram B, et al. A PSMA-targeted theranostic agent for photodynamic therapy. J Photochem Photobiol B Biol.; 2017;167:111-6.

Wang X, Tsui B, Ramamurthy G, Zhang P, Meyers J, Kenney M E, et al. Theranostic Agents for Photodynamic Therapy of Prostate Cancer by Targeting Prostate-Specific Membrane Antigen. Mol Cancer Ther. 2016;15:1834-44.

Wang J, Liu Q, Zhang Y, Shi H, Liu H, Guo W, et al. Folic Acid e Conjugated Pyropheophorbide a as the Photosensitizer Tested for In Vivo Targeted Photodynamic Therapy. J Pharm Sci; 2017;106:1482-9.

Watanabe R, Hanaoka H, Sato K, Nagaya T, Harada T, Mitsunaga M. Photoimmunotherapy Targeting Prostate-Specific Membrane. Nat Rev. 2018;56:140-5.

Kumar D, Shan L, Zhang Y. Nanoparticles in photodynamic therapy: An emerging paradigm. Adv Drug Deliv Rev.; 2008;60:1627-37.

Heine M, Freund B, Nielsen P, Jung C, Reimer R. High Interstitial Fluid Pressure Is Associated with Low Tumour Penetration of Diagnostic Monoclonal Antibodies Applied for Molecular Imaging Purposes. PLoS One. 2012;7:e36258-e36258.

Israeli R S, Powell C T, Corr J G, Fair W R, Heston W D W. Expression of the Prostate-specific Membrane Antigen. Cancer Res. 1994;54:1807-11.

Bostwick D G, Pacelli A, Blute M, Roche P, Ph D, Murphy G P. Prostate Specific Membrane Antigen Expression in Prostatic Intraepithelial Neoplasia and Adenocarcinoma A Study of 184 Cases. 1998;2256-61.

Kiess A P, Minn I, Vaidyanathan G, Hobbs R F, Josefsson A, Shen C, et al. (2S)-2-(3-(1-Carboxy-5-(4-211At-Astatobenzamido)Pentyl)Ureido)-Pentanedioic Acid for PSMA-Targeted-Particle Radiopharmaceutical Therapy. J Nucl Med. 2016;57:1569-75.

Haberkom U, Eder M, Kopka K, Babich J W, Eisenhut M. New Strategies in Prostate Cancer: Prostate-Specific Membrane Antigen (PSMA) Ligands for Diagnosis and Therapy.

Clin Cancer Res. 2016;22:9-15.

Kratochwil C, Bruchertseifer F, Giesel F L, Weis M, Verburg F A, Mottaghy F, et al. 225Ac-PSMA-617 for PSMA-Targeted a-Radiation Therapy of Metastatic Castration-Resistant Prostate Cancer. J Nucl Med. 2017a;57:1941-5.

Kratochwil C, Giesel F L, Stefanova M, Bene M, Bronzel M, Afshar-oromieh A, et al. PSMA-Targeted Radionuclide Therapy of Metastatic Castration-Resistant Prostate Cancer with 177Lu-Labeled PSMA-617. J Nucl Med. 2017b;57:1170-7.

Stefflova K, Li H, Chen J, Zheng G. Peptide-based pharmacomodulation of a cancer-targeted optical imaging and photodynamic therapy agent. Bioconjug Chem. 2007;18:379-88.

Zheng G, Li H, Zhang M, Lund-katz S, Chance B, Glickson JD. Low-Density Lipoprotein Reconstituted by Pyropheophorbide Cholesteryl Oleate as Target-Specific Photosensitizer. Bioconjug Chem. 2002;13:392-6.

Chandran S S, Banerjee S R, Mease R C, Pomper M G, Denmeade S R. Characterization of a targeted nanoparticle functionalized with a urea-based inhibitor of prostate-specific membrane antigen (PSMA). Cancer Biol Ther. 2008;7:974-82.

Chang S S, Reuter V E, Heston W D W, Bander N H, Grauer L S, Gaudin P B. Five Different Anti-Prostate-specific Membrane Antigen (PSMA) Antibodies Confirm PSMA Expression in Tumor-associated Neovasculature 1.1999;3192-8.

Jin C S, Overchuk M, Cui L, Wilson B C, Bristow R G, Chen J, et al. Nanoparticle-enabled selective destruction of prostate tumor using MRI-guided focal photothermal therapy. Prostate. 2016; 76: 1169-81.

Castanares M A, Copeland B T, Chowdhury W H, Liu M M, Rodriguez R, Pomper M G, et al. Characterization of a novel metastatic prostate cancer cell line of LNCaP origin. Prostate. 2016;76:215-25.

Chen Y, Gryshuk A, Achilefu S, Ohulchansky T, Potter W, Zhong T, et al. A Novel Approach to a Bifunctional Photosensitizer for Tumor Imaging and Phototherapy. 2005;1264-74.

Kiess A P, Minn I, Chen Y, Hobbs R, Sgouros G, Ronnie C, et al. Auger Radiopharmaceutical Therapy Targeting Prostate-Specific Membrane Antigen. J Nucl Med. 2015;56:1401-7.

J. Shi, T. W. B. Liu, J. Chen, D. Green, D. Jaffray, B. C. Wilson, F. Wang G Z. Transforming a Targeted Porphyrin Theranostic Agent into a PET Imaging Probe for Cancer. Theranostics. 2011;1:363-70.

Serrell E C, A B, Pitts D, D M, Hayn M, D M, et al. Review of the comparative effectiveness of radical prostatectomy, radiation therapy , or expectant management of localized prostate cancer in registry data. Urol Oncol Semin Orig Investig.; 2017;1-10.

Fakhrejahani F, Madan R A, Dahut W L. Management Options for Biochemically Recurrent Prostate Cancer. Curr Treat Options in Oncol (2017). Current Treatment Options in Oncology; 2017;18:1-19.

Liu T, Wu L Y, Choi J K, Berkman C E. In vitro targeted photodynamic therapy with a pyropheophorbide-a conjugated inhibitor of prostate-specific membrane antigen. Prostate. 2009;69:585-94.

Liu T, Wu L Y, Berkman C E. Prostate-specific membrane antigen-targeted photodynamic therapy induces rapid cytoskeletal disruption. Cancer Lett [Internet]. Elsevier Ireland Ltd; 2010;296:106-12.

Nagaya T, Nakamura Y, Okuyama S, Ogata F, Maruoka Y, Choyke P L, et al. Near-Infrared Photoimmunotherapy Targeting Prostate Cancer with Prostate-Specific Membrane Antigen (PSMA) Antibody. Mol Cancer Res. 2017;15:1153-62.

Nakajima T, Mitsunga M, Bander N, Heston W D, Choyke P L, Kobayashi H. Targeted, Activatable, In Vivo Fluorescence Imaging of Prostate-specific Membrane Antigen (PSMA)-positive Tumors Using the Quenched Humanized J591 Antibody-ICG Conjugate. Bioconjug Chem. 2012;22:1700-5.

Watanabe R, Hanaoka H, Sato K, Nagaya T, Harada T, Mitsunaga M. Photoimmunotherapy Targeting Prostate-Specific Membrane. 2018;56:140-5.

Minchinton A I, Tannock I F. Drug penetration in solid tumours. Nat Rev Cancer. 2006;6:583-92.

Liu G, Banerjee S R, Yang X, Yadav N, Lisok A, Jablonska A, et al. A dextran-based probe for the targeted magnetic resonance imaging of tumours expressing prostate-specific membrane antigen. Nat Biomed Eng.; 2017;1.

Neuman B P, Ei J B, Castanares M, Chowdhury W H, Chen Y, Mease R C, et al. Real-time, Near-Infrared Fluorescence Imaging with an Optimized Dye/Light Source/Camera Combination for Surgical Guidance of Prostate Cancer. Clin Cancer Res. 2015;21:771-80.

Rowe S P, Gage K L, Faraj S F, Macura K J, Toby C, Gonzalez-roibon N, et al. 18F-DCFBC PET/CT for PSMA-Based Detection and Characterization of Primary Prostate Cancer. J Nucl Med. 2015;56:1003-10.

Szabo Z, Mena E, Rowe SP, Plyku D, Nidal R, Eisenberger M A, et al. Initial Evaluation of [18 F]DCFPyL for Prostate-Specific Membrane Antigen (PSMA)-Targeted PET Imaging of Prostate Cancer. Mol Imaging Biol. 2015;17:565-74.

Giesel F, Will L, Lawal I, Lengana T, Kratochwil C, Vorster M, et al. Intra-individual comparison of 18 F-PSMA-1007 and 18 F-DCFPyL PET/CT in the prospective evaluation of patients with newly diagnosed prostate carcinoma: A pilot study. J Nucl Med. 2018; 59:1076-1080.

Hofman M S, Hicks R J, Maurer T, Eiber M. Prostate-specific Membrane Antigen PET: Clinical Utility in Prostate Cancer, Normal Patterns, Pearls, and Pitfalls. Radiographics. 2018;38:200-17.

Zhang J, Jiang C, Longo J P L, Azevedo, R B, Zhang H, and Muehlmann, L A, Acta Pharmaceutica Sinica B, 2017;8:137-146.

Chen Y, Dhara S, Banerjee S R, Byun Y, Pullambhatla M, Mease R C, et al. A low molecular weight PSMA-based fluorescent imaging agent for cancer. Biochem Biophys Res Commun. 2009;390:624-9.

Cheng Y, Prusoff W H. Relationship Between the Inhibition Constant (Ki) and the Concentration of Inhibitor which Causes 50 per cent Inhibition (ISO) of an Enzymatic Reaction. Biochem Pharmacol. 1973;22:3099-108.

Wilson B C, Firnau G, Jeeves W P, Brown K L, Burns-McCormick D M. Chromatographic analysis and tissue distribution of radiocopper-labelled haematoporphyrin derivatives. Las Med Sci. 1988;3:71-80.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A theranostic probe comprising a compound of formula (I):
P-L-T (I)
wherein:
P is a porphyrin-based photosensitizer;
L is a peptide linker; and
T is a urea-based PSMA-targeting ligand; and
pharmaceutically acceptable salts thereof.

2. The theranostic probe of claim 1, wherein the photosensitizer further comprises a radiometal.

3. The theranostic probe of claim 2, wherein the radiometal is selected from the group consisting of ⁶⁴Cu, ⁶¹Cu, ⁶⁷Cu, ¹¹¹In, ⁸⁹Zr, and ⁶⁸Ga.

4. The theranostic probe of any of claims 1-3, wherein the photosensitizer is capable of multimodal fluorescence imaging and radioimaging.

5. The theranostic probe of claim 4, wherein the radioimaging is positron emission tomography (PET) imaging or single photon computed emission tomography (SPECT) imaging.

6. The theranostic probe of claim 1, wherein the peptide linker comprises a D-peptide sequence comprising from about 5 to about 15 D-amino acids.

7. The theranostic probe of claim 6, wherein the D-peptide sequence comprises about nine amino acids.

8. The theranostic probe of claim 7, wherein the D-peptide sequence is GDEVDGSGK.

9. The theranostic probe of claim 1, wherein the urea-based PSMA-targeting ligand comprises the following chemical moiety: 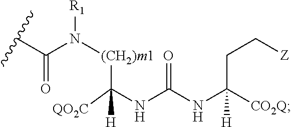
wherein:
m1 is an integer selected from 1, 2, 3, 4, 5, 6, 7, and 8;
Z is tetrazole or —CO₂Q;
each Q is independently selected from the group consisting of hydrogen, substituted or unsubstituted straight-chain or branched C₁-C₈ alkyl, substituted or unsubstituted aryl, and a protecting group; and
R₁ is selected from the group consisting of hydrogen, substituted or unsubstituted straight-chain or branched C₁-C₈ alkyl, and substituted or unsubstituted aryl.

10. The theranostic probe of claim 1, wherein the probe comprises a compound of formula (Ia): 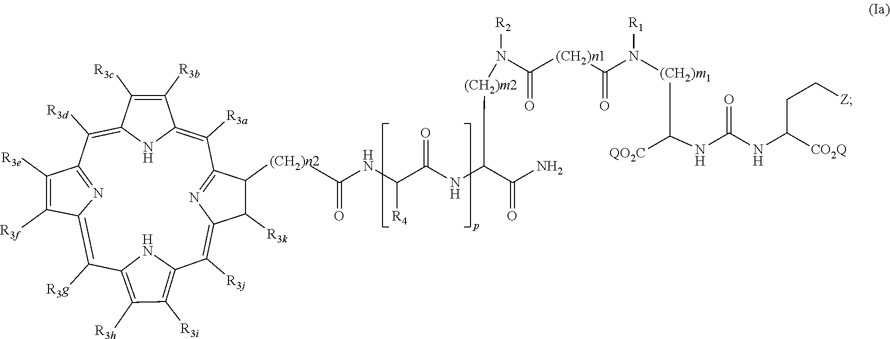
wherein:
m1 and m2 are each independently an integer selected from the group consisting of 1, 2, 3, 4, 5, 6, 7, and 8;
n1 and n2 are each independently an integer selected from the group consisting of 1, 2, 3, 4, 5, 6, 7, and 8;
p is an integer selected from the group consisting of 5, 6, 7, 8, 9, 10, 11, 12, 13, 14, and 15;
Z is tetrazole or —CO₂Q;
each Q is independently selected from the group consisting of hydrogen, substituted or unsubstituted straight-chain or branched C₁-C₈ alkyl, substituted or unsubstituted aryl, and a protecting group; and
R₁ and R₂ are each independently selected from the group consisting of hydrogen; substituted or unsubstituted straight-chain or branched alkyl, substituted or unsubstituted aryl;
R₃ₐ, R_{3b}, R_{3c}, R_{3d}, R₃ₑ, R_{3f}, R_{3g}, R₃ₕ, R₃ᵢ, R₃ⱼ, and R₃ₖ are each independently selected from the group consisting of substituted or unsubstituted straight-chain or branched C₁-C₈ alkyl, substituted or unsubstituted C₁-C₈ alkenyl, substituted or unsubstituted aryl, wherein the aryl can be substituted with one or more substituent groups selected from substituted or unsubstituted straight-chain or branched C₁-C₈ alkyl, hydroxyl, C₁-C₈ alkoxyl, amino, cyano, carboxyl, halogen, —SO₃⁻, and oxo;
or R₃ₐ and R_{3b}, R_{3c} and R_{3d}, R_{3d} and R₃ₑ, R_{3f} and R_{3g}, R_{3g} and R₃ₕ, R₃i and R₃ⱼ, and R₃ⱼ and R₃ₖ can together form a 5- to 6-member carbocyclic ring along with the porphyrin ring, which can be substituted with one or more substituent groups selected from substituted or unsubstituted straight-chain or branched C₁-C₈ alkyl, hydroxyl, C₁-C₈ alkoxyl, amino, cyano, carboxyl, halogen, and oxo; and
each R₄ is independently selected from the group consisting of substituted or unsubstituted straight-chain or branched C₁-C₈ alkyl, substituted or unsubstituted C₁-C₈ alkenyl, substituted or unsubstituted aryl, —(CH₂)ₙ₃—OR₅, —(CH₂)ₙ₄—CO₂R₆, —NR₇R₈, —SR₉, —SeR₁₀, substituted or unsubstituted cycloheteroalkyl, substituted or unsubstituted aryl, substituted or unsubstituted arylalkyl, substituted or unsubstituted heteroarylalkyl, and substituted or unsubstituted heteroaryl;
wherein n3 and n4 are each independently an integer selected from the group consisting of 1, 2, 3, 4, 5, 6, 7, and 8; and
R₅, R₆, R₇, R₈, R₉, and R₁₀ are each independently selected from the group consisting of hydrogen and substituted or unsubstituted straight-chain or branched C₁-C₈ alkyl; and
pharmaceutically acceptable salts thereof.

11. The theranostic probe of claim 10, wherein the probe has the following chemical structure:

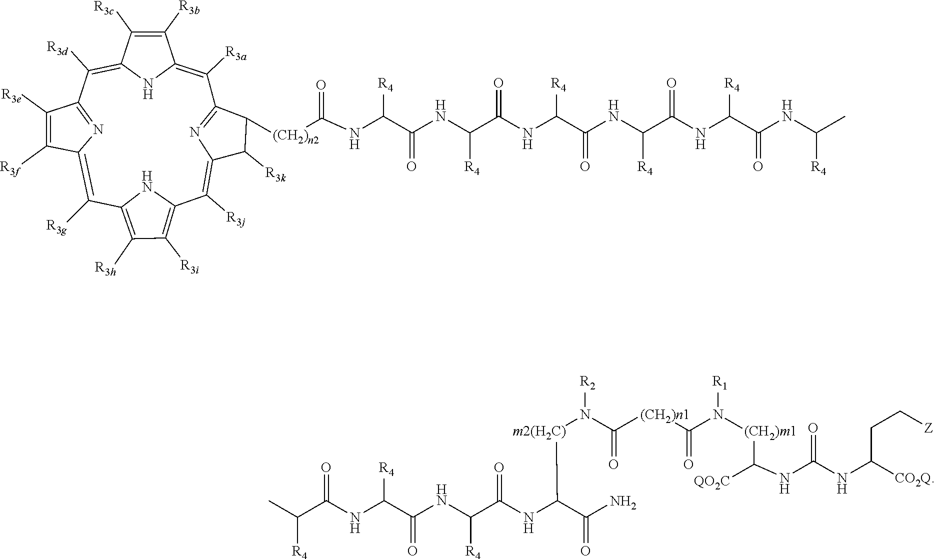

12. The theranostic probe of claim 11, wherein the probe has the following chemical structure:

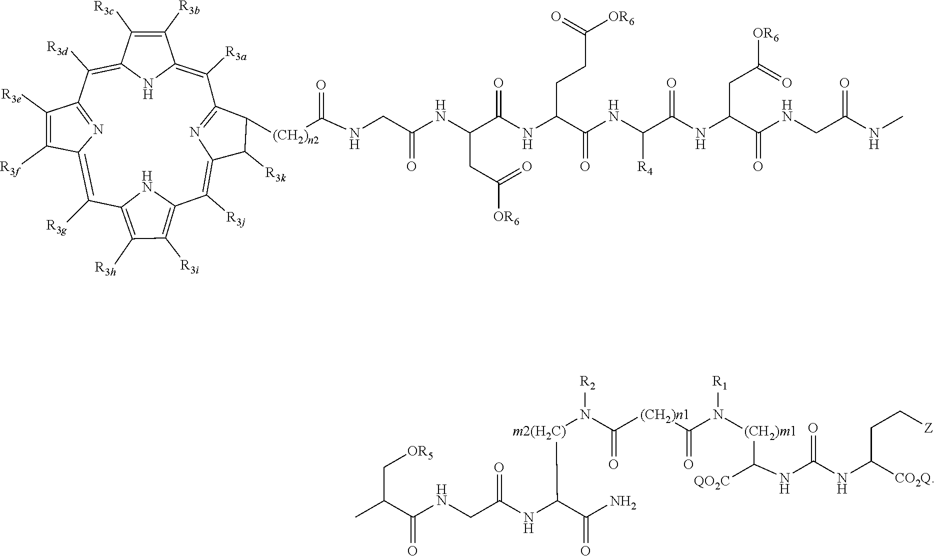

13. The theranostic probe of claim 12, wherein the probe has the following chemical structure:

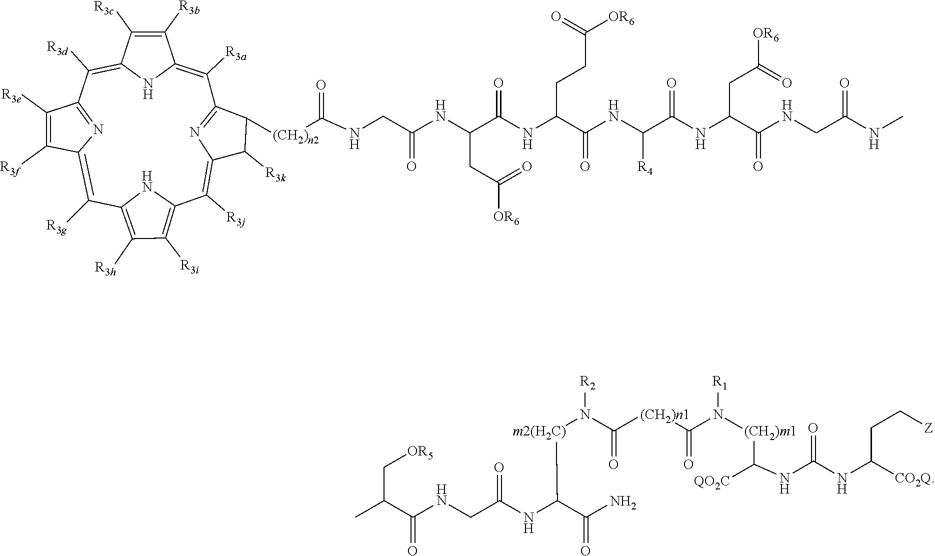

14. The theranostic probe of claim 13, wherein the probe has the following chemical structure:

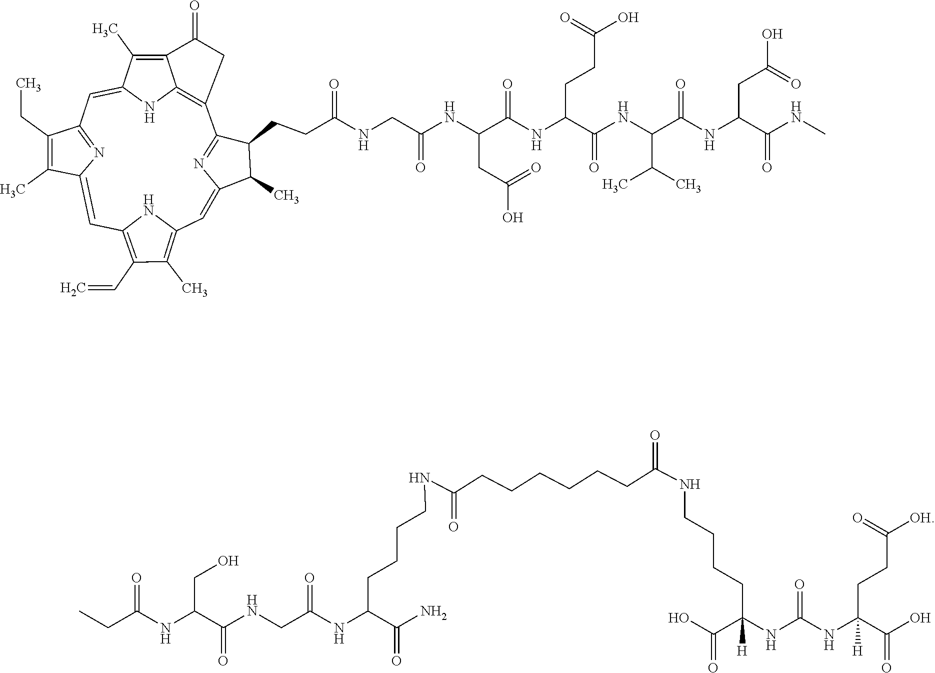

15. The theranostic probe of any of claims 6-14, wherein the photosensitizer further comprises a radiometal.

16. The theranostic probe of claim 14, wherein the radiometal is selected from the group consisting of ⁶⁴Cu, ⁶¹Cu, ⁶⁷Cu, ¹¹¹In, ⁸⁹Zr, and ⁶⁸Ga.

17. A method for treating or imaging one or more PSMA expressing tumors or cells, the method comprising contacting the one or more PSMA expressing tumors or cells with an effective amount of a theranostic probe of any of claims 1-16.

18. The method of claim 17, wherein the one or more PSMA-expressing tumor or cell is selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof.

19. The method of claim 17, wherein the one or more PSMA-expressing tumor or cell is a prostate tumor or cell.

20. The method of claim 17, wherein the one or more PSMA-expressing tumor or cell is in vitro, in vivo, or ex vivo.

21. The method of claim 17, wherein the one or more PSMA-expressing tumor or cell is present in a subject.

22. The method of claim 21, wherein the subject is human.

23. The method of claim 17, wherein the method results in inhibition of the tumor growth.

24. The method of claim 17, further comprising taking an image.

25. The method of claim 24, wherein the taking of an image comprises positron emission tomography (PET) or single photon computed emission tomography (SPECT).
